(19) United States
(12) Patent Application Publication
(10) Pub. No.: US 2025/0140419 A1
(43) Pub. Date: May 1, 2025

(54) GAME THEORY TOKEN MISALIGNMENT DETECTION AND REMEDIATION DEVICE

(71) Applicant: LogicMark, Inc., Louisville, KY (US)

(72) Inventors: Chia-Lin SIMMONS, Louisville, KY (US); Rafael SAAVEDRA, Louisville, KY (US); Peter WILLIAMS, Louisville, KY (US)

(73) Assignee: LogicMark, Inc., Louisville, KY (US)

(21) Appl. No.: 18/919,216

(22) Filed: Oct. 17, 2024

Related U.S. Application Data
(60) Provisional application No. 63/595,043, filed on Nov. 1, 2023.

(51) Int. Cl. G16H 50/30; A63F 13/217; G16H 10/60

(52) U.S. Cl. CPC G16H 50/30; A63F 13/217; G16H 10/60

(57) ABSTRACT

A system, apparatus, and method using game theory and machine learning a system, device and method that uses game theory and machine learning to monitor a person under care.

### BACKGROUND

### Field of the Disclosure

Aspects of the disclosure relate in general to using game theory and machine learning to monitor a person under care.

### Description of the Related Art

In traditional infrastructure technology environments, Personal Emergency Response Systems (PERS), also known as Medical Emergency Response Systems, allow persons to call for help in an emergency by pushing a button.

One example system is a two-way voice communication pendant that allows a person to call for assistance anywhere around their home. Personal emergency response devices make aging in place and independent living a possibility for persons under care. The personal emergency response device allows a person to remain connected with loved ones and emergency services through an existing landline telephone.

### SUMMARY

Embodiments include a system, device and method that uses game theory and machine learning to monitor a person under care.

In one aspect, a system monitors a person under care by a stakeholder. The system comprises a transceiver, a non-transitory computer-readable storage medium, and at least one processor. The transceiver is configured to receive a plurality of tokens from a plurality of environmental sensors configured to monitor the person under care. Each of the tokens comprises a detected data set representing behaviors of the person under care in an environment. Each of the behaviors is represented by a multi-dimensional feature set forming part of a health care profile for the person under care. The non-transitory computer-readable storage medium is configured to store a digital twin of the plurality of tokens from the plurality of environmental sensors. The digital twin comprises a dynamic tokenized representation of the multi-dimensional feature set forming part of a health care profile of the person under care in the environment. The at least one processor is configured to evaluate the digital twin of the plurality of tokens from the plurality of environmental sensors as part of game to optimize an optimum set of configuration of the plurality of environmental sensors, and change a state of the plurality of environmental sensors based on the game or notify the stakeholder.

In another aspect, a system to monitor a person under care by a stakeholder, comprises a transceiver, a non-transitory computer-readable storage medium, and at least one processor. The transceiver is configured to receive a plurality of tokens from a plurality of environmental sensors configured to monitor the person under care. Each of the tokens comprises a detected data set representing behaviors of the person under care in an environment. Each of the behaviors is represented by a multi-dimensional feature set forming part of a health care profile for the person under care. The non-transitory computer-readable storage medium is configured to store a strategy in a game of optimizing the plurality of environmental sensors configured to monitor the person under care. The strategy has at least one strategy equilibrium. The at least one machine learning processor is configured to apply the strategy to the plurality of tokens from the plurality of environmental sensors as part of the game to optimize an optimum set of configuration of the plurality of environmental sensors, and change a state of the plurality of environmental sensors based on the game or notify the stakeholder.

### BRIEF DESCRIPTION OF THE DRAWINGS

To better understand the nature and advantages of the present disclosure, reference should be made to the following description and the accompanying figures. It is to be understood, however, that each of the figures is provided for the purpose of illustration only and is not intended as a definition of the limits of the scope of the present disclosure. Also, as a general rule, and unless it is evident to the contrary from the description, where elements in different figures use identical reference numbers, the elements are generally either identical or at least similar in function or purpose.

FIG. 1 is a block diagram illustrating pattern detection system.

FIG. 2 is an illustrative example of pattern detection based on one or more sensor data incorporating tokens.

FIG. 3 is an illustrative example of differing games based on one or more sensor data incorporating pattern matching.

FIG. 4 is a further illustrative example of differing games based on one or more sensor data incorporating machine learning.

FIG. 5 is a further illustrative example of differing games involving configuration of other sensors.

FIG. 6 is an illustrative example of differing games based on sensor data incorporating machine learning and pattern matching.

FIG. 7 is an illustrative example of differing games based on sensor data incorporating predictive pattern matching.

FIG. 8 is an illustrative example of differing games for evaluation of responses.

FIG. 9 is an illustrative example of differing games for evaluation of responses.

FIG. 10 is an illustrative example of multiple sensor data sets evaluated for detection of patterns and bevokens.

FIG. 11 is an illustrative example of multiple sensor data sets evaluated for detection of patterns and bevokens.

FIG. 12 is an illustrative example of multiple sensor data sets, patterns and bevokens integrated with care processing.

FIG. 13 is an illustrative example of bevoken evaluation for game determined responses where state is quiescent.

FIG. 14 is an illustrative example of bevoken evaluation for game determined responses where state is not quiescent.

### DETAILED DESCRIPTION

Aspects of the present disclosure include a system, device and/or method described herein that uses game theory and machine learning, including generative AI, to identify and develop representations of potential and/or probable incentive misalignments and/or other attributes, events, alerts, behaviors, patterns and/or data sets within systems and/or with stakeholders of those systems.

### Game Theory Applied to Care and Wellness

A person under monitoring (PUM) in an environment, for example a Sensor Enabled Environment (SEE) can have multiple sensors, devices and/or systems monitoring them and/or the environment state in the context of their care and well-being. An aspect of this monitoring is the configuration of each of these sensors, devices and/or systems so as to, in a timely manner, accurately represent the state of the PUM and/or their environment.

One of the challenges is the configuration of each of the sensors, devices and/or systems so as to optimize the timeliness and accuracy of this monitored state, particularly without reverting to a central control system, where incomplete, inaccurate or sparse data can lead to responses that are inappropriate. This is especially the case where a sensor indicates an event that could be represent the PUM being in danger, for example having a fall, when in fact the PUM simply tripped with no ill effects. This often causes false positives with attendant costs, stress and unnecessary resource deployment.

One aspect of the system described herein is the use of various strategies to ensure that the data representing the state of the PUM and their environment is accurate and as far as possible complete. In this manner the appropriate response may be initiated in an efficient and effective manner, considering, for example, both the response resource provider and the PUM.

In some embodiments, game theory may be employed to establish an accurate and timely representation of the state of the PUM and their environment. This application of game theory can include the use of machine learning techniques, including generative AI, in support of data analysis, strategy determination, classification and/or game deployment.

One aspect of this approach is the reduction in reliance on predefined, anticipated and/or pre planned use cases, which the typical development method for current monitoring systems and much software and systems in generally the case

The use of games, based on tokenized data sets comprising patterns based on observed and/or measured sensed data provides an alternative approach to system design and implementation that is not limited by the definition of use cases that are anticipated to occur.

### Sensors

A game may be deployed to represent the configurations and outcomes that a sensor, device and/or system may employ whilst undertaking monitoring of, for example, a PUM in their environment, for example a SEE, to create one or more data set.

In some embodiments one or more games may be operated by one or more hardware processors in or accessible to a sensor, device and/or system where the game comprises players that may include the sensor itself, other sensors, devices and/or systems, stakeholders and/or AI/ML, including agents, for example agents of an LLM, instantiations. In some embodiments where for example each sensor is operating one or more game, these games may include representations of the data sets generated by such sensor when monitoring a PUM and/or other stakeholders in their environment, for example a SEE. These data sets can be represented by one or more tokens and as such games can involve these tokens.

In some embodiments, a game outcome may include the transfer of one or more tokens to one or more sensors, devices and/or systems. For example, this can include tokens that can configure a sensor, device and/or system. Other tokens may represent behaviors, patterns and/or other data sets that in whole or in part represent the state of a PUM in an environment that is being monitored, for example a SEE. Games can include connections, for example represented by one or more tokens, that are communicated to one or more other sensors, devices and/or systems such that multi step games between sensors, devices and/or systems can be deployed.

In some embodiments, there may be multiple capabilities for communicating between sensors, devices and/or systems. This can include out of band communications, tokenized communications, synchronous and/or asynchronous communications and the like. Such communications can include prioritized messaging, such that the receiving sensors, device and/or system respond to such a message in a time sensitive manner. For example, if one or more sensor, device and/or system sends such a message to another one or more sensor, device and/or system, the receiver may then reconfigure operations in a manner previously specified in response to that message with a priority that such specifications determine. In this manner an immediate reconfiguration of one or more sensors, devices and/or systems may be executed with minimal delay. In some embodiments, the message may be in the form of a token.

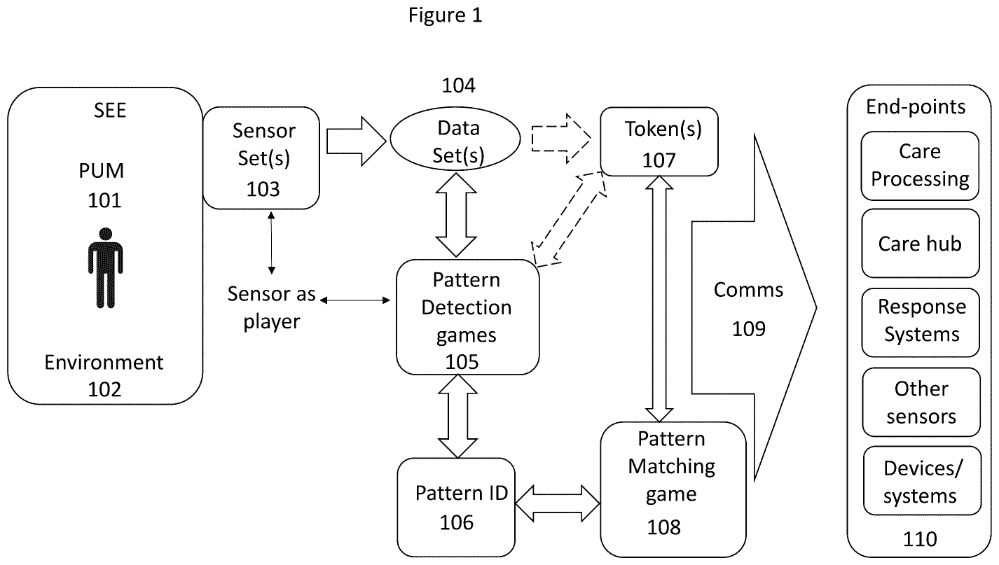

FIG. 1 illustrates a person under monitoring (PUM-101) in an environment (102), where one or more sensors (103) are operating and generating one or more data sets (104). These data sets (104) can be evaluated by one or more games, for example pattern detection games (105) where one or more sensor may operate as a player in such games. These games may include one or more pattern identification (106), where known and previously identified patterns are the outcomes of the games. Data sets (104) and pattern detection games (105) may, for example using a token module, generate one or more tokens (107) representing such data sets (104) and/or patterns that have been detected and identified. The pattern matching games may employ one or more tokens in one or more games to match those tokens, including those that represent data sets (107), with identified patterns (106) using pattern matching games (108). These outcomes can then be communicated (109) to one or more end points, including for example, care processing, care hubs, response systems, other sensors and/or devices and systems (110). In some embodiments, devices/systems (110) may include one or more ML/AI module, where for example such patterns form apart of one or more training sets.

A further aspect is determining the allocation of sensor, device and/or system resources amongst a set of such sensors, devices and/or systems. The determination of complimentary sensor, device and/or system arrangements may, in part be determined by one or more games. For example, if a sensor detects movement, then a complimentary, co-located and/or other sensor may detect sound, such that if the first sensor detects a movement, and the second sensor detects a sound simultaneously, the games may use Shapley values to determine the relative payoffs and/or enable communications between the sensors, devices and/or systems that are competing to provide data that would match, for example an event token or other representation of that occurrence, including for example a pattern.

### Tokens

In some embodiments where a token may represent the identity of a set of source sensors, devices and/or systems and/or the data generated by such sensors, devices and/or systems, these tokens may be directed to one or more other sensors, devices and/or systems for evaluation, processing and/or use.

In some embodiments, there are various tokens that represent various states and situations pertaining to the wellness and care of a PUM in an environment. For example, there may be Time of Day (ToD) tokens which are quantization's of a PUM's behaviors over the period of a day. For each PUM these tokens may have differing start, finish and duration clock times, though in aggregate they represent the overall behaviors expressed in a temporal manner for a PUM. As such an individual PUM may have specific ToD tokens that represent their particular behavior. Such a ToD token may comprise the set of patterns that the sensor data and potentially other data sets represents.

For example, based on a 24 hour clock cycle this may include:
Sleep
Wake
Breakfast
Morning set
Lunch
Afternoon set
Dinner
Evening set
Retire
Sleep

Each of these ToD tokens may be instantiated multiple times, for example a PUM may opt to have an early breakfast and a late breakfast, or may have a sleep ToD token in the middle of the afternoon. These base behavior tokens can support the sensors, devices and/or systems to manage these time periods in the context of the PUM's behaviors which may vary according to clock time, but represent a pattern and/or sequence when expressed as ToD tokens.

Each of the ToD tokens may form part of the behavior sets of other parties involved with the PUM, for example other stakeholders, such as carers, friends and/or family, where their respective interactions are represented by bevokens, including ToD tokens.

This approach supports one or more games representing such interactions where for example a PUM, represented by the one or more bevokens representing their behaviors and one or more stakeholders, represented by the bevokens representing their behaviors in such interactions, may form part of a game representing such interactions. For example, such a game as prisoners dilemma or similar may represent these interactions and as such one or more sensors, devices and/or systems may be employed to provide a response, stimulus, suggestion, action and/or event that may benefit the care, wellness, health and/or safety of the PUM.

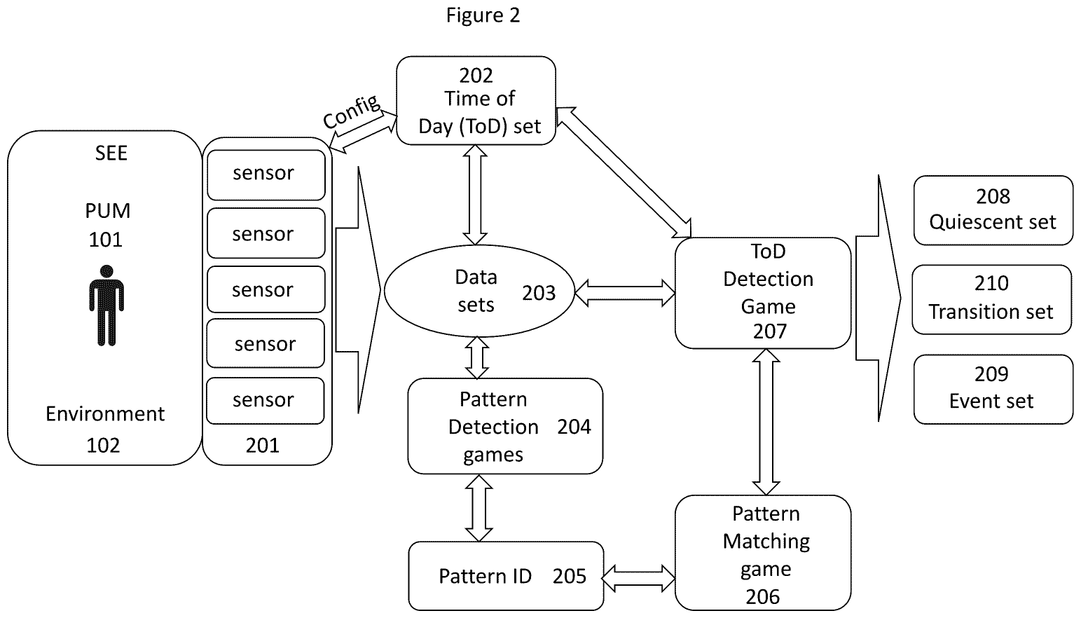

FIG. 2 illustrates an example embodiment of a sensor enabled environment-SEE-(102) where a PUM (101) is monitored by a set of sensors (201) which generate one or more data sets (203). These data sets are evaluated, at least in part, by one or more Time of Day configuration sets (202), which can include the configuration of the one or more sensors (201). The sensor data sets (203) inform, in combination with the ToD sets one or more ToD detection games (207). The data sets (203) are for example, simultaneously evaluated by one or more pattern detection games (204) in combination with pattern identification systems (205), communicating with one or more pattern matching games (206). The outputs of these games may be a set of tokens or other data representations, being either quiescent (208), event (209) or transition (210) types representing the state of the PUM (101) in the environment (102).

A further set of tokens are known as behavior tokens, termed Bevokens. In some embodiments, these are classified into two broad types, the first is quiescent sets and the second are event sets.

Quiescent sets can include, for example, behaviors represented as bevokens:
Exercise set
Nap set
Waste set
Body clean set
Entertainment set
Social set
Reading set
Writing set
Communications set
Regular medicine set
Hobby set
Work set
Specialist activity set
And the like.

For each PUM there may be specific bevokens that represent that PUM's behaviors. This can include data sets from one or more sensors, devices and/or systems that represent those behaviors which includes the one or more patterns representing such data. Such bevokens may, for example, include one or more thresholds or other parametrizations, such that if these are exceeded or fall out of the range expressed by that bevoken, the state of the PUM is identified as having changed.

In some embodiments, a bevoken may comprise a set of patterns which are tokenized representations of data sets of one or more sensors. For example, a data set from one or more sensors may display a particular pattern and as such that pattern is tokenized and forms part of a bevoken. This in combination with other patterns from one or more other sensors may comprise the set of patterns represented by that bevoken.

In some embodiments each PUM's Bevoken may incorporate a set of patterns that represent the data sets that are the representation as determined by the one or more sensors, devices and/or systems of that PUM's behaviors. For example, a standardized ToD token may be initially used, for example a morning token, which may then have a relationship with, for example a reading bevoken, representing the PUM activity of reading a book, newspaper or other source in the morning. For example, there may be simultaneous tokens representing the behaviors of a PUM, for example a ToD token may be operating, for example afternoon sets in parallel with a nap Bevoken.

Bevokens generally represent the quiescent state of the PUM in that the activities and ToD represented by these Bevokens conforms to the observed PUM activities that match the one or more patterns represented by the data sets degenerated by one or more sensors, devices and/or systems.

However, there can be multiple situations where one or more sensors detect an event, which may also be represented by one or more tokens. These event tokens represent a change in state of the PUM, and may include both care and wellness events and/or other events.

Event tokens can include, for example:
Health visit set
Medical activity set
Trip set
Fall set
Breathing impairment set
Impact set
Shopping
Visiting set
Travel
And the like.

All of these bevokens can comprise data sets from one or more sensors that area employed in monitoring a PUM and their environment, and may include sensors, devices and/or systems that are embedded within an environment and/or are worn and/or carried by a PUM.

The types of tokens, including Bevokens are not limited to ToD, quiescent and/or event tokens and may include specialist tokens, for example those representing particular health and wellness treatments, such as physical therapy. There may also be one or more alert tokens, where information is communicated amongst the parties, such as one or more stakeholders. This can include tokens which initiate an action by, for example, one or more sensors, devices, systems and/or stakeholders in any arrangement.

In some embodiments, one or more games can incorporate one or more Bevokens (behavior tokens), where for example game payoffs may represent prioritization of those Bevokens. For example, a payoff for a game can be weighted towards a particular outcome, for example a value that is higher than the payoff for an alternative outcome, and that higher outcome is represented by a particular Bevoken, providing a weighting function.

In some embodiments, a bevoken is a token that represents a behavior of a person where such behaviors comprise one or more patterns. The person may be, for example, a person under monitoring (PUM) or at least one stakeholder in a care village. The bevoken may be comprised of one or more patterns detected, at least in part, by one or more sensors, devices and/or systems, including those of a SEE

One aspect is the various inputs to one or more sensors operations within the context of monitoring a PUM in an environment. Attributes of the sensor and its operation may include granularity and/or resolution of the sensing, battery life and availability, configuration of the sensor, availability of the sensing elements to other sensors, devices and/or systems, priority of requests and/or provision of sensor data to one or more sensors, devices and/or systems, privacy and/or other identity-based specifications and the like.

In many current systems these attributes are formed into rule sets which are not only rigid, but often are unable to anticipate situations for which the rules have not been configured which are often defined in terms of use cases.

The use of game theory significantly improves the flexibility and responsiveness of the one or more sensors, devices and/or systems, their relationships to one or more other sensors, devices and/or systems that are, in whole or in part, configured to monitor a PUM in an environment, for example a SEE. The use of one or more games where the data from a sensor, device and/or system, configuration of that sensor, device and/or system and their operation can form part of one or more games where a range of potential and actual players may be involved. This can include the sensor, device and/or system itself, one or more other sensors, devices and/or systems, the PUM and/or other stakeholders and/or third party systems in any arrangement.

One consideration is the use of games to determine, in whole or in part, when to acquire, process and/or store data from one or more sensors, devices and/or systems. This can include the relationship between a sensor and a repository, such as an elastic repository, where data from the sensor may be stored, potentially for an extended period outside the normal configuration of that sensor, for example when a game managing such a relationship, for example one where a trigger external to the sensor originating the data, has a payoff that configures the relationship between the sensor and the repository.

For example, an event may be detected by a sensor, such as when a PUM has tripped or other similar health, wellness and/or safety event, such that the trigger is part of a game that is in operation by that and other co-located sensors, such that each of the sensor generated data is retained in an appropriate repository. For example, this data may then be evaluated, processed and/or used by one or more sensors, devices and/or systems, which can include one or more AI/ML system, including generative AI, to determine the context and accuracy of the initial sensor data that triggered the game and consequent storage of the data. In some situations such data may be made available to one or more stakeholders such as an EMT or other stakeholders in support of the well-being, health and/or safety of the PUM in their environment.

The use of games to determine, in whole or in part, the actions, configurations and/or responses of one or more sensors, devices and/or systems, involved in the monitoring of a PUM in a SEE, provides a context responsive approach to optimizing how these entities can provide the most effective health, wellness and safety care support, for example as a comprehensive set of verified, accurate and timely data, for the monitoring of a PUM in an environment. The adaptive abilities of the use of games, in that the strategies employed by a player of a game can create outcomes that integrate the sensed data representing the state of a PUM in an environment that in turn can generate one or more response strategies, potentially involving further games, creating a flexible and situationally responsive system that is not dependent on anticipated use case driven rules and specifications. This enables the overall care, wellness and/or safety monitoring and response enablement for a PUM in order to, in part or in whole, avoid the constraints, false positives and other restrictions of current use-case based systems.

One aspect of this approach is the use of games involving a plethora of sensors, where for example a set of sensors may be extended by the inclusion of further sensing of one or more sensors, devices and/or systems. For example, sensor 1 detects a signal that exceeds one or more thresholds or parameters of that sensor such that the sensor, using one or more communications methods, including for example tokens, communicates with another sensor, where for example the second sensor operates to provide one or more data set that can be evaluated to validate or not the initial data of the first sensor.

One aspect is the accurate determination of the state of an environment using multiple sensors, devices and/or systems where, for example, a sensor is designated as an edge device, that is a sensor that is configured to detect a change in state of the environment and communicate that change of state event to one or more other sensors, devices and/or systems. However, such an event may occur if the sensor is malfunctioning, has an external stimulus, such as an insect, obscuring the sensor and the like, and as such one or more games may be operating to, at least in part, establish the validity of the sensor operations. For example, multiple sensors, acting as players, may be triggered by the original event for the first sensor and may then be configured to provide data sets, potentially in the form of tokens, that form part of the operating game, to determine the accuracy of the original event. The outcome of this game may then initiate further communications with other sensors, devices and/or systems depending on this outcome. Such an approach can reduce false positives and extraneous sensor data.

In some embodiments there may be multi-player games where each sensor, device and/or system operating to monitor a PUM in an environment, for example a SEE, can compete in such games to provide the relevant and/or optimum configuration for one or more other sensors, devices and/or systems so as to validate an event detected by a first sensor through configuration of one or more other sensors, devices and/or systems. This competition may result in a payoff in the form of a bevoken representing a particular event, expressed as a pattern and/or behavior. For example, confirmation that the monitored event is the PUM tripping over a carpet and the like.

In some embodiments there may be one or more games that are operating to support the selection of the optimum configurations for one or more sensors, devices and/or systems that enable those entities to detect and/or confirm an event, pattern and/or behavior in the an accurate, timely and/or efficient manner.

In some embodiments data sets from other devices and/or systems connected to and/or representing such other devices, including refrigerators, smart TV's, smart speakers, smart phones, worn and/or carried devices, HVAC systems, entry/exit systems, vehicles, home security systems (including cameras and motion detectors) and the like may form part of the data sets that can be used by the one or more care village systems, including as data which forms part of one or more game.

In some embodiments one or more games may be invoked to, at least in part, configure such other devices and/or systems such that the data sets generated may form part of a game and/or contribute to data sets that in combination with data sets from sensors, devices and/or systems monitor a PUM in a Sensor Enabled Environment (SEE).

In some embodiments, an individual data set generated by such home automation appliances and/or devices may only have relevance when expressed in a statistical format, such as when a data set is an outlier for the normal, or quiescent behavior of such a device. In some embodiments, the data from these devices may be evaluated by one or more systems to establish these quiescent behaviors, which may be represented as tokens using similar or same formats to those described herein. If a data set is outside of calibrated and/or configured thresholds, then, for example, an event or alert token may be created. These quiescent and event or alert tokens may form parts of games being undertaken, for example, by the systems monitoring these home devices.

In some embodiments, one or more AI/ML systems, including generative AI, such as LLM's may be employed to evaluate the one or more data sets generated by such home automation and/or appliances, including for example using such data to train an LLM to identify, in whole or in part, when such data represents a state that is outside the quiescent state of those data.

### Trust Relationships Between Sensors

One key aspect is the relationship between sensors, devices and/or systems whereby a sensor establishes a relationship with, for example other sensors, devices and/or systems such as those that are collocated and/or are calibrated and/or configured to monitor a PUM in a sensor enabled environment (SEE). This can include the use of one or more machine learning algorithms, including neural networks, deep learning, generative AI and the like that can analyze data sets generated by the one or more sensors, devices and/or systems to establish the accuracy and/or reliability of such data sets. These analytics, expressed in some embodiments as metrics, for example accuracy, deviation, reliability and the like, may then be used by one or more games operating in one or more sensors, devices and/or systems including sets thereof to establish further metrics such as trust, truth and other similar metrics that are payoffs from those games. For example, such an approach may enable a sensor to establish a trust relationship with another sensor, such that from both a security and performance basis the data generated by such other sensor may be trusted as a ground truth by the first sensor. Further the strategies employed by the second sensor involved in the game, or set thereof, where both sensors are, directly or indirectly acting as players in such game, provides a security and surety factor which may be used to determine, at least in part, whether an imposter, substitution or other malign sensors, devices and/or systems are operating.

In some embodiment, sensors, devices and/or systems may rely, in whole or in part, on specifications that represent the configuration of other sensors, devices and/or systems. Such specifications, in whole or in part may be represented by one or more tokens, where such tokens may form part of one or more games in which the sensors, devices and/or systems are participating. For example, each sensor may store and/or access the configuration of one or more other sensors. These configuration data sets may be represented by one or more tokens. For example, certain sensing capabilities and/or their outcomes may be represented by a token, for example, a particular sensitivity may require a certain resource such as battery power. These tokens may then form part of a game, for example a game to provide validation of an event detection by one or more other sensor.

In some embodiments, such a game may be played out by a set of sensors, with differing tokens representing differing configurations of one or more sensors to establish the optimum configurations for each of the sensors involved to satisfy the objective of the game.

In some embodiments a set of sensors may have insufficient processing to undertake the evaluation of differing configuration specifications for that sensor and/or other sensors. In this example, a digital twin, or set thereof, may be instantiated such that each of the one or more sensors and the potential differing configurations are deployed therein, where the digital twin includes additional processing and/or other resource that is not available to the physical sensors, devices and/or systems. Such as approach can support the evaluation of such configuration options for the one or more sensors. This can include the operation of one or more games designed to support the identification of the optimum set of configurations for the one or more sensors. These configurations can then be deployed to the operating sensors. For example, if the configuration of sensor 1 is A, B or C and sensor 2 is D, E or F, the determination of which configuration that optimizes the outcome can depend on the selection of a configuration of sensor 1. In this manner a game can be employed to evaluate the optimum response to the selection of, for example configuration A for sensor 1 by the selection of, for example, configuration D by sensor 2. This can include determining the Nash equilibrium for the selection of the configurations of these sensors, where for example B and F represent the optimum choice for monitoring the current state of the PUM in an environment.

In multiple sensor environments, particularly those where many of the devices have multiple sensing capabilities, including where there may be a duplication of capabilities, albeit at differing granularities, one or more games, for example those employing mixed strategies may be employed to determine the optimum set of sensors to be employed, which can then, for example using further games, be configured to provide the optimum sensing of the PUM in a SEE.

In some embodiments there may be a number of devices, sensors and/or systems that generate data sets for monitoring a PUM in a SEE, each of these may have one or more calibrations and/or configurations, representing multiple possible states and characteristics of these sensors, devices and/or systems. Representing these as strategies in a game where, there can be multiple mixed strategy equilibria, can involve numerous equations with multiple unknowns and as such employing of one or more ML/AI systems to, at least in part determine which of these possibilities can reduce the complexity of establishing which of the equilibria represent the set combinations that optimize the efficacy, timeliness, efficiency and/or accuracy of the data sets generated by these sensors, devices and/or systems. In some situations there may be multiple equilibria, each of which can have desirable characteristics related to the current and potentially future state of a PUM in an environment. These multiple equilibria may then be deployed within one or more digital twin which can then invoke one or more AI/ML module to establish the potential for each of these related to the PUM. One or more further systems, for example a care hub or care processing system may then select and prioritize these equilibria and calibrate and/or configure the one or more sensors, devices and/or systems. For example there may be 4 equilibria (A,B,C,D), and the care hub and/or care processing systems may for example initially deploy B, and arrange the order of further deployments as C,D, A, where such deployments can, at least in part, be determined by the one or more patterns and/or behaviors of the PUM.

One aspect of this is approach is establishing the trustworthiness of the data representing the configuration data of the one or more other sensors, such that a sensor, device and/or system can rely on that data being accurate, timely and true. In some embodiments a sensor may have a set of configuration data for another one or more sensors which that sensor has retained, which may need to be updated. In this example the first sensor may use a confirmation game, where each of the other sensors with which the first sensors has a trust relationship, for example those involved in the monitoring of a specific PUM in a specific environment, provides a tokenized set of configuration data to a game that employs a consensus algorithm, for example a byzantine algorithm, to establish which set of configuration data is accurate and up to date.

Although one sensor can provide a second sensor with configuration data, the provision of this data without appropriate security and privacy protection can create a serious risk. The use of tokenized data and the operations of these games can mitigate and/or avoid such risk.

The application of AI/ML techniques, including generative AI, including LLM's, to the data sets provided by one or more sensors, devices and/or systems supports the determination, by for example a care hub and/or care processing system, of the sensors own behaviors. For example, a sensor in an environment, such as a house, room and/or car, has certain characteristics that may vary depending on the environment conditions. For example, the temperature, humidity, presence of a person, time of day and the like. In this manner the sensor data will represent these variations, often as minor variations of the data output of the sensor and/or as an EMF field representing the operations of the sensor. For example, one or more AI/ML technique may be employed to identify theses variations in such a manner so as to fingerprint a sensor, both for security and/or for fault detection.

The same techniques may be employed to determine the appropriate game structure and/or strategy for a game, for that sensor when interacting with one or more other sensors.

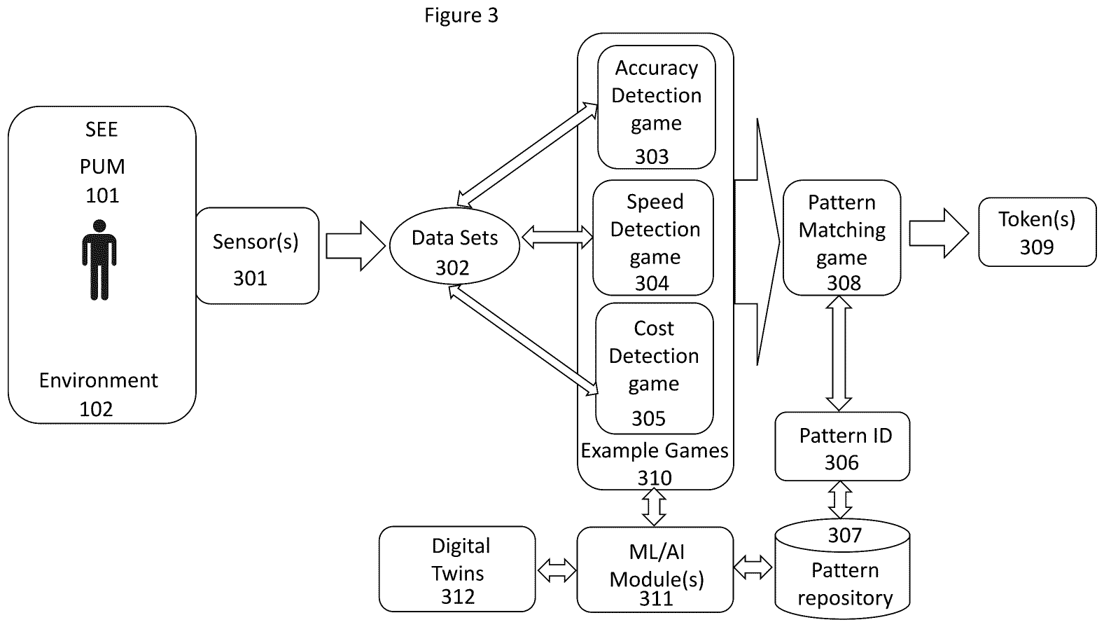

FIG. 3 illustrates an environment (102) that includes a PUM (101), where a set of sensors (301) generate data sets (302). These data sets and the sensors from which they are generated may form part of one or more example games (310). For example, there may be games with differing variables, payoffs and outcomes, for example accuracy detection game (303) where the payoffs for that game are determined by the accuracy of the date sets. Another game involving the same sensors and data sets generated by those sensors, may be a speed detection game (304), where the speed of detection of an event or other data set results in a payoff for the one or more sensors. There may also be a cost detection game (305), where the costs, for example expressed as the resources used by the sensor, such as energy, for example from a battery, time, processing and the like determines the payoff, with for example the least use of the costs providing the highest payoff. These games and their outcomes may be evaluated as part of a pattern matching game, where the games, strategies employed and the outcomes are evaluated by a pattern matching game (308) to ascertain any known or detected patterns. The pattern identity store (306) may be used by the pattern matching game (308) to determine whether the pattern is recognized and as such has an appropriate payoff and/or outcome The outcomes of this pattern matching (308) and pattern identification (306) may be stored in one or more pattern repository (307). In some embodiments, a pattern matching game may generate one or more token (309) representing the outcome of the game, for example the recognition of an identified pattern. In some embodiments pattern matching may operate in parallel with one or more machine learning and AI systems (311), including the use of digital twins (312).

In some embodiments, one or more sensors, devices and/or systems may employ one or more games where the outcomes of those games can be configuration of another sensor, device and/or system. For example, such a game may be deployed when a first sensor data set reaches a threshold or other parameter, and prior to generating an alert or event, instantiates a game, which based on the data set has an outcome that sends a configuration communication to another sensor. The second sensor, depending, at least in part, on the configuration of that sensor, may accept this configuration change and generate a data set which is communicated to the first sensor. This data set may then be used to in whole or in part confirm or refute the first sensor data sets, resulting in an event or alert being generated or not. In some embodiments, this communication may be in the form of secure tokens

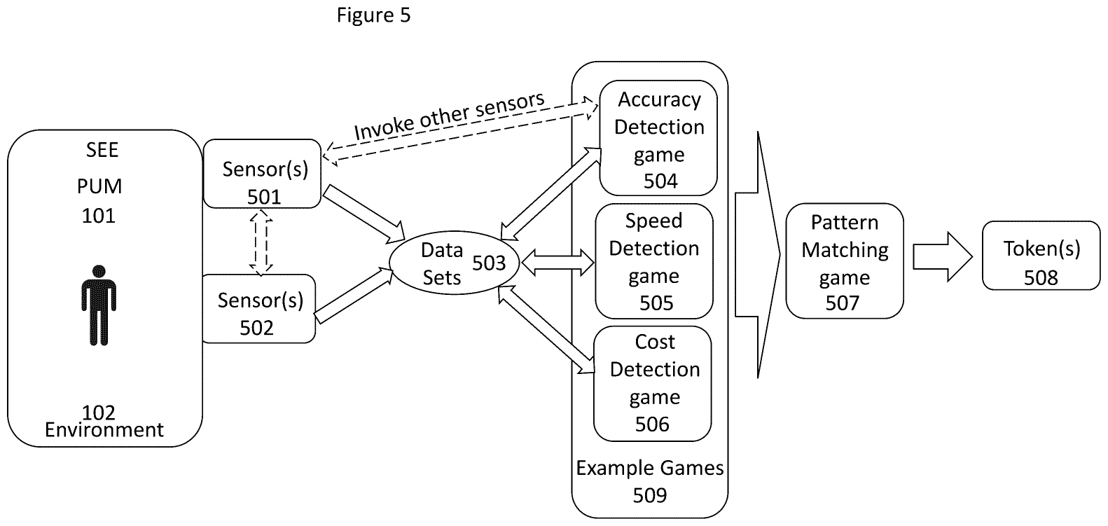

FIG. 5 illustrates an example of an environment (102) with a PUM (101) where one or more sets of sensors (501,502) are operating to generate one or more data sets (503). In this example, there are three example game (509) types operating, accuracy detection (504), speed detection (505) and cost detection (506), which in turn provide outcomes and payoffs that are evaluated by one or more pattern matching games (507), which in turn can generate one or more tokens (508). In this example the data sets generated by a first sensor set (501) which then forms part of an accuracy detection game, can be configured to invoke further sensor sets (502) to generate further data sets which in combination with the sensor sets of the first sensor set (501) form a part of a strategy which results in the first sensor set, acting as a player in the game, achieving an outcome that generates a payoff. For example, the data sets of the second sensor set (502) may provide additional data that increases the accuracy of the data set of the first sensor set (501), so that the first sensor set (501) achieves an outcome resulting in a payoff.

### Trust Games

In some embodiments, when human stakeholders are participating in one or more games and those stakeholders perceive the other players as also being human, relationships of trust may be set up. For example, if a stakeholder acts in a game with a strategy that does not create outright benefit for them at the expense of another player, the other player may be more inclined to choose a strategy that has a similar benefit for themselves. This equivalence, or reciprocity, has been observed in trust game behavior research, for example Berg et al 1995 and Burks et al 2003.

One aspect of this is when a first player initiates a strategy where they invest a resource, for example time, financial, emotional support and/or other quantifiable behavior, for example one represented by a bevoken, which in turn encourages other stakeholders as players in the game to reciprocate.

Many games representing trust have assumptions involving trading current value for future value, often based on monetary expressions. For example, in the domain of the care village, there may be a wider set of transactions, such as resources, finance, wellness and care, emotional responses, time, availability, collaboration and the like which may be represented as value expressions.

In some embodiments, the use of tokens as a form of quantized patterns and/or behavior representations instantiated as the expressions of these values within one or more games enables those games to create payoffs and/or outcomes that are expressed as further token sets representing further patterns and/or behaviors. In this manner a game may be played by a number of players where the payoffs for that game are, for example, a PUM or other stakeholder pattern and/or behavior representations. For example, if a set of players is undertaking a game configured to identify whether a set of data from one or more sensors, devices and/or systems represents a variation of a pattern and/or behavior that indicates that a PUM is transitioning from one quiescent state to another state, where such states may be represented as bevokens, the payoff may be expressed as a token representing that transition, which can be determined from a set of potential transitions, by the strategies employed by the players.

In some embodiments a sensor, device and/or system may be configured to have one or more thresholds pertaining to the measurements the sensor is making. If the values of those measurement is within, for example, a range of values, which can include a degree of uncertainty, for example represented in the form of a metric for the threshold, then the sensor may generate a token, which for example represents a state that neither matches a quiescent state, for example as a bevoken, nor is a match for an event state, for example an event token. In this indeterminate state, the sensor may generate both types of token, quiescent and event, though both with an assigned parameter value that indicates the uncertainty of the measurements. In this example, a game involving the sensor, device and/or system as a player, may have a requirement that only a single token can be used. In this example, the sensor, device and/or system may directly or indirectly invoke an ML/AI module which will act as a player representing the sensor, device and/or system where such an ML/AI player may then act to use both tokens in a game. In this manner the uncertainty of the sensors measurements is represented so that differing payoffs may be evaluated. One advantage of this approach is that the payoffs of the game can include, for example, an action where the differing strategies for the game result in an evaluation of the indeterminate token to a determinate state, that is quiescent or event, without resorting to fixed and inflexible threshold based decisions. For example, the action that is the payoff of the game may to invoke one or more other sensors to validate, or not, the original measurements of the initial sensor.

One form of game that may be deployed, for example on one or more sensors, can involve payoffs that reward, for example, energy consumption and/or conservation versus sensing measurements and their timing. For example, if a sensor operates for an extended period using the available energy supply, for example a battery, then the rate of sensing can impact that period of operations. This can include the timing of these measurements, including continuous, sequential or sampled. A game that has payoffs that reward the optimization of sensing versus operating period may be deployed on and/or for one or more sensors. In some embodiments this can include a game of this type operating on and/or for a number of sensors, such that the measurements are distributed across the multiple sensors in a manner that conserves the battery supply of each sensor whilst providing continuous or near continuous measurements. In this example, there may also be one or more systems, such as a care hub and/or care processing system that can change the configuration of the sensor in light of the circumstances, for example where a PUM is experiencing an event.

In the example of sensors, devices and/or systems, the tokens generated by these entities may be in a form that supports their evaluation, through for example data normalization, such that the efficiency of the operations is enhanced through this normalization. This can apply to data equivalence and tokenization thereof and may include of tokenization of one or more behaviors, for example represented by bevokens.

In some embodiments, reciprocal behavior with non-normalized data sets and/or equivalence of behaviors and/or stakeholder intentions can be represented by game strategies and/or game outcomes. For example, the operation of sequential and/or simultaneous games may enable evaluation of reciprocity, such as for example if a message is sent to one stakeholder, for example as an outcome of a game and such message, for example a move in that game by a stakeholder, may influence another player involved in that game. This can be particularly useful where the stakeholder intentions are not fully stated and the moves of the game are intended, at least in part, to vary the behaviors of the stakeholders, generally for the benefit of the PUM and/or to minimize any disadvantage to one or more stakeholders. For example, this can include the use of Bayesian games, where for example the behaviors of a stakeholder, including a subset thereof representing for example their typical behaviors for a specific context, can be represented in the game as their priors, which can be considered as their private information and as players these priors can be used to form posterior beliefs, which can be represented as their potential behaviors, determined through their strategies deployed within the game, where such payoffs are part of the set representing the potential behaviors available to the players within the context. One aspect of this approach is determining the beliefs of a stakeholder as a player in relation to the beliefs of other stakeholders, also as players, resulting in determining the potential behaviors of these stakeholders.

In some embodiments such beliefs and consequent actual and/or predicted behaviors amongst sets of stakeholders may be used to train one or more AI/ML systems such that one or more models may be instantiated, and consequently avoiding describing a hierarchy of all beliefs of the stakeholder and their behaviors in a hierarchical form. This enables the use of an AI/ML model to, using potentially a sparse data set, represent the potential behaviors of a set of stakeholders to a context, and configure a game with payoffs that incentivise certain behaviors that, at least in part benefit a PUM and/or mitigate any risks and safety issues for that PUM and/or the other stakeholders.

In some embodiments, a specific context, including for example a SEE with a PUM domiciled therein, where the PUM is undertaking, for example a set of behaviors that are quiescent, can form part of a common prior for one or more Bayesian games, where the behaviors of one or more other stakeholders are considered.

One aspect is the representation of “completeness” using one or more games, such that the monitoring is capable of representing a “complete” picture of the state of a PUM in an environment. This can be extended to any of the stakeholder involved, including individuals and/or organizations, though the completeness of knowledge by the system of all stakeholders is unlikely, and to a large degree unnecessary, in that the behaviors evidence their intentions and actions within any particular context and/or situation. Establishing the veracity and validity of data sets representing those behaviors as generated by the one or more sensors, devices and/or systems, can in whole or in part, be established through the use of one or more games, where such sensors, devices and/or systems act as players in such games. In this manner the requisite balance between time, accuracy, resources, reliability, privacy, identity, costs, risks, safety and/or other characteristics may be achieved, in whole or in part, through the one or more strategies deployed by such sensors, devices and/or systems. In some embodiments one or more ML/AI module may operate with and/or for such sensors, devices and/or systems and/or may operate as an independent player, for example representing one or more care village system, such as care processing.

In some embodiments, sensors, devices and/or systems may employ one or more decision trees, which can be representations of game outcomes as payoffs. For example, if a player, for example a sensor, has a choice as to possible action the sensors may initiate, these can be decision nodes. The payoffs of the game are represented by terminal nodes, where each of these nodes represents an action that a sensor may undertake. For example, in some embodiments a sensor may have the following terminal nodes as choices of the decision tree.
informing Care hub and/or Care Processing of an event (may include type and/or probability)
initiating a configuration change of sensor itself
initiating a configuration change of one or more other sensors
initiate differing games (including Digital Twin based games).
identification of event types with probability
adoption of one or more AI/ML model
invoking one or more AI/ML system, including generative AI (LLM)
Forming part of a RAG for an LLM

In some embodiments extensive form games may be deployed that involve a set of players that have some foreknowledge through the moves of other players when making their moves within a game. This is particularly the case where, for example one or more sensors makes a move in a game based on, for example an event detected by such sensors that exceeds one or more thresholds of the configuration of that sensor.

This can also be the case where one or more stakeholders undertake one or more behaviors, including patterns, that provide another one or more stakeholder with foreknowledge of that behavior in a game, resulting in differing strategies being employed by that stakeholder.

In some situations, where a set of sensors are operating as players in an extensive form game, each of the sensors may initiate their move in the game based, at least in part, on the data set available to that sensor. This data set can include, for example, the configuration of the sensor, the configuration of other sensors, the data generated by the first sensor and/or data sets generated by one or more other sensors. Such data sets may include configuration data, sensors status data (such as battery life, communication availability) and/or other contextual data, such as environmental conditions.

In some embodiments, these game moves may not be in any chronological order, but rather they may be made in an order determined by the data available to the one or more sensors. In some embodiments, the moves of one or more sensors may be undertaken by a proxy of the sensor, for example a module that has sufficient processing, storage and/or other resources, including the data sets of the sensor, to undertake participating in such a game. For example, in some embodiments, such a module may be an AI/ML module that represents one or more sensors in such a game. Such a module may also initiate one or more digital twins to evaluate the potential moves and strategies available in the context of that game.

### Care Processing

One aspect of the monitoring of a PUM and their environment, including the deployment of multiple sensors, devices and/or systems is the sets of data produced, that may need to be evaluated. These data sets may be a combination of raw and/or tokenized data in any arrangement. The tokenized data may be encrypted and/or in the clear. However, in many such embodiments, the data can be represented by secure token, in part, to protect the privacy of one or more stakeholders, including the PUM.

In some embodiments, an aggregator, such as a care hub and/or care processing system may intermediate between the data sources, for example, sensors, devices and/or systems and the one or more recipients of the data sets generated by such sources. In some embodiments one or more care hub and/or care processing systems may be the recipient of and/or interact with such data.

For example, in many circumstances a care hub and/or care processing system may initially act to verify or validate the data received from one or more sensors, devices and/or systems to, at least in part, ascertain that the data is an as accurate possible representation of the current situation. For example, this can include evaluating multiple sensor, device and/or system data sets so as to validate that the data set of one or more sensors, devices and/or systems is verified and/or validated by other sensors, devices and/or systems involved in the monitoring of an environment. This may include management of the one or more configurations of the one or more sensors, devices and/or systems, such that, for example an initial data set representing, for example an event that, for example, represents a potentially detrimental impact on a PUM may be verified and/or validated.

This can include the use of one or more games by the care hub and/or care processing systems where the incoming data sets are represented by, for example, tokens, which form part of the unfolding game and the payoffs of such games may be further tokens representing configuration and/or other data that can be communicated to one or more sensors, devices and/or systems.

This can include alignment of differing data sets on a time line, where for example each data set, which in some embodiments may be represented by one or more tokens, provides a differing perspective on the current situation of the environment under monitoring. For example, a sensor capable of detecting an image, may provide a change of vertices of a PUM, whilst a second sensor provides a signal derived from audio data that could be an impact, whilst a third sensor, for example a haptic sensor provides a signal indicating an impact, all of which are coincident with data from a worn or carried sensor set providing a token indicating an acceleration and change in altitude.

A care hub and/or care processing system may include one or more games where such data sets are represented by tokens where that game, which may include sub-games, has a set of payoffs based on the combination of the tokens representing an event, for example an event bevoken, such as a fall. Such an event bevoken may include one or more calls to action, including alerts that can be communicated to one or more stakeholders, including third parties.

In some embodiments contested or contradictory data sets, including for example those represented as tokens, may be evaluated by one or more systems, including care hub and/or care processing systems. These contradictory tokens may be evaluated in the form of a game, where the outcome is an event bevoken. For example, if a single sensor generates a token indicating an event and other collocated other sensors generate tokens that do not indicate such an event, then the game outcome may be a quiescent bevoken. In some embodiments, the game may include an outcome which is a further token, for example a token, that includes the contradictory data sets and the outcome of the game, which can then be stored in one or more repository, where for example one or more AI/ML system may be trained on such data sets.

In some embodiments a care hub and/or care processing system may invoke one or more digital twins in which games are played with the tokens representing the data generated by the sensors, devices and/or systems. In this manner the games operating in the one or more digital twins, using for example differing games, strategies, payoffs and/or outcomes may provide differing outcome possibilities, for example if a contradictory token is generated, the digital twin, which may include one or more AI/ML systems as a player of the game, may determine a differing configuration for the one or more other sensors co located with the originating sensor and may communicate such a configuration, represented by a token, to a care hub and/or care processing systems for further evaluation and/or deployment.

One aspect of the care village is the deployment of processing capabilities, particularly those embodied in devices that are worn or carried and/or those that have battery-based power supplies.

In some embodiments, a care hub and/or care processing system may have sufficient processing power so as to be able to undertake multiple games simultaneously, including invoking one or more digital twins, which may include one or more AI/ML modules configured, for example, as players of these games.

In some embodiments, the care hub and/or care processing systems may instantiate one or more AI/ML modules which may act as a player, representing the care hub and/or care processing systems in games that are deployed and unfolding involving one or more sensors, devices and/or systems.

This can include, for example, integration of hierarchical games where the care hub and/or care processing systems operates as player in multiple games, including those games that are hosted on or by a sensor, device and/or system. For example, a care hub and/or care processing system may invoke on one more preferences for resources used and/or applied to one or more sensors, devices and/or systems with which it is operating.

In some embodiments, the application of generative AI, including LLM's, can support evolutionary game theory games, whereby a game is evolved in light of the strategies employed by the AI, so as to create a complete efficient game-based representation of an unfolding situation based on the monitoring of a PUM in an environment. Part of this approach is the development of strategies by the generative AI, potentially using causal machine learning to determine in the most efficient and timely manner the most accurate representation of the context and situation involving the PUM under monitoring where there are one or more contradictory, incomplete, sparse, partial or other data sets, that for example can be represented by tokens.

In some embodiments, an LLM may be trained on data sets from the one or more sensors, devices and/or systems in SEE where a PUM is domiciled. These data sets may represent the context and current situation of the PUM, however the LLM may generate outcomes that have limited accuracy. The use of consensus games, where the LLM plays against itself can improve the overall accuracy and consistency of the LLM outcomes. This approach can include the use of the Nash Equilibrium as a number of games are played, and can also include the use of quantal response functions which involve using differing payoff's for these games. The use of multiple LLM, where for example one LLM is using consensus games with Nash equilibrium and a second LLM is determining payoff's for quantal responses, can lead to highly accurate and efficient determination of the context and situation of a PUM, including where there may be sparse data, multiple stakeholders and/or other complicating factors.

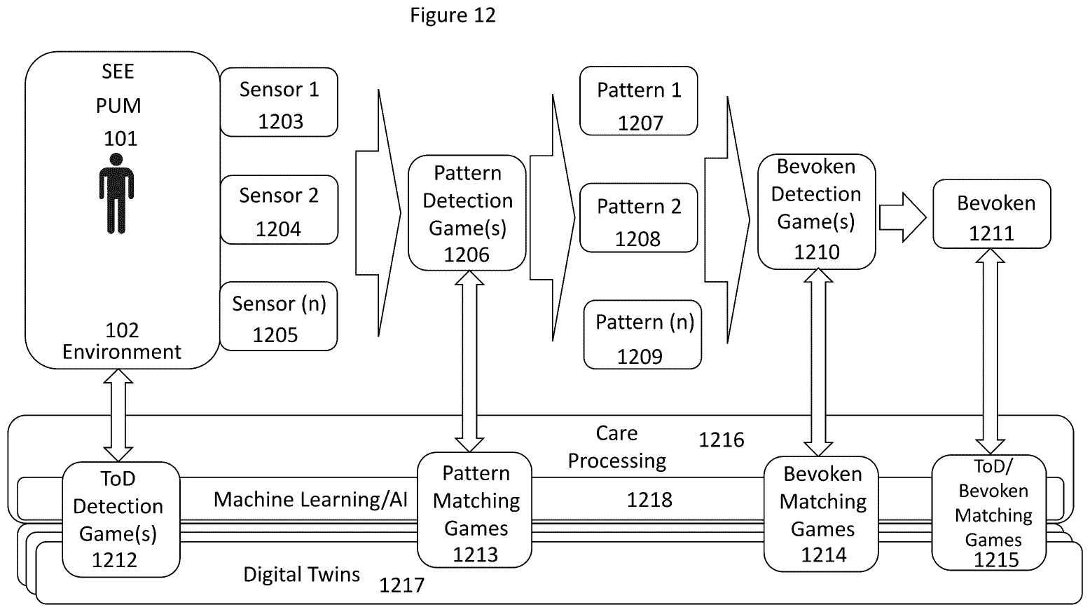

FIG. 12 illustrates an environment (102) in which a PUM (101) is monitored by sets of sensors, including sensor set 1 (1203), sensor set 2 (1204) and sensor set (n) (1205). These sensor sets and the data sets they generate are evaluated by one or more pattern detection games (1206), which may then identify patterns for the one or more sensor sets, for example pattern 1 (1207) for sensors set 1 (1203), pattern 2 (1208) for sensor set 2 (1204) and pattern (n) (1209) for sensor set (n) (1205). These patterns (1207,1208,1209) may then be further evaluated by one or more bevoken detection games (1210) so as to generate one or more bevokens (1211) that represent these patterns as identified as such. The environment (102) and PUM (101) may contemporaneously be evaluated by one or more ToD detection games, where the Time of Day patterns are detected and matched. The care processing systems (1216), which can include one or more machine learning and/or AI modules (1218), including for example one or more LLM, may operate with, for example pattern matching games (1213), bevoken matching games (1214) and/or ToD bevoken matching games (1215) in any arrangement to determine, at least in part the outcomes of these games. In some embodiments these outputs and other data sets may be communicated to one or more digital twins (1217), which may in combination with one or more machine learning and/or AI system, provide predictive analytics and outcomes that are managed by the care processing systems. (1216)

In some embodiments techniques such as first order derivatives, representing state changes expressed as metrics, which can include the use of vectors, may be represented as game tokens. This can include options derived, at least in part, from the game strategies and the data sets employed therein.

In some embodiments the use of games to, at least in part, determine the weightings and/or tensors for one or more generative AI, for example an LLM, may be deployed. For example, games using Nash or quantal response equilibrium may be used, including as sets of games using consensus algorithms, such as byzantine, can be employed to generate weighting and attentions that can be used in an encoder for an LLM.

One aspect of the care processing systems is the generation of responses to the data sets generated by the one or more sensors, devices and/or systems. These responses can involve specifications, configurations, instructions and/or other commands and/or communications to the sensors, devices and/or systems and/or to one or more stakeholders, including the PUM.

This can involve the deployment of one or more games, which may include one or more response strategies. These games may include those that are intended to evaluate the impact of the outcomes generated in by the responses. This can include evaluation of multiple strategies for outcomes and/or responses with differing weightings and/or priorities assigned to those outcomes and/or responses, either explicitly in the payoffs of these games and/or as weightings for the outcomes and/or responses that are evaluated by further systems, such as a care village risk module. For example, the use of quantal response functions to vary the payoffs, that for example represent the potential impacts of a response can be used, for example, in digital twins to determine, at least in part the potential impact on a PUM prior to that response being initiated.

In some embodiments a game identification module may have available a common set of games that are designed for specific events, for example when a PUM enters a room, undertakes a pattern and/or behavior and/or experiences an event, for example, where multiple sensors, devices and/or systems, including for example care processing systems contribute to and/or participate in one or more game. For example, one or more event states represented, for example, as a sequence of events based, at least in part on one or more detected patterns, for example, represented by one or more tokens, can be represented by one or more strategies deployed in one or more games, such that each pattern and/or behavior can be represented by one or more game with one or more strategies. For example, each strategy can have differing outcomes and/or responses, potentially representing one or more stakeholder intentions. This can include the use of games specifically for negotiation, where players, including sensors, devices and/or systems, stakeholders, care village systems, for example care processing, have one or more objectives that are, in whole or in part, in opposition, contradiction, orthogonal and/or conflict. This approach can support conflict resolution and/or consensus building and may include operations of one or more digital twins, where differing strategies, players, payoffs, equilibria and the like may be deployed to identify opposing, contradictory, orthogonal and/or conflicting interests.

### Machine Learning

One aspect of the system is the use of machine learning (ML), including generative IA, neural networks, deep learning and/or other ML/AI techniques in the selection of one or more games and/or game parameters, including strategies and/or payoffs, players and/or rules and game rules and structures.

Such ML/AI techniques may be employed to determine strategy options for one or more games, such that appropriate strategies can be deployed.
This can include the use of ML/AI for game creation from existing games and/or game strategies.

In some embodiments one or more games can be setup as multi-player/multi-agent games, where ML/AI models can be used as one or more of the players/agents in the game. This can be applied to real-life systems, interacting live with sensors, other devices and/or other stakeholders, and/or in simulated environments, where historical and/or predicted datasets can be used along with ML/AI agents to test decisions and their outcomes. In such environments Digital Twins can be used, to form, at least in part, a prediction system.

In some multi-agent environments ML/AI models can be used to negotiate with other parties based on predefined strategies and other parameters, such as PUM's preferences, for example those specified in a HCP (Health Care Profile). These systems can learn from past interactions, adapt to different negotiation styles, and make decisions that maximize the expected outcome. ML/AI techniques such as reinforcement learning can be applied to these scenarios to help ML/AI agents learn optimal strategies through, for example, trial and error. This approach can be effective in dynamic environments where the other players/agents' strategies and the outcomes are not fully known in advance.

Other techniques, such as Meta-learning, can be employed to develop models that can quickly adapt and learn from new game scenarios. This is particularly beneficial in dynamic environments where the model needs to continuously update its strategies based on changing conditions or new information, which can be the case in scenarios when a PUM's condition changes and/or when the regulations, service rules, costs and/or other parameters of one or more stakeholders change. Meta-learning focuses on developing models that can quickly adapt and learn from new patterns, tasks, behaviors and/or environments. In the context of game theory, meta-learning can be applied to build adaptive models that can continuously update their strategies based on evolving conditions and/or new information in interactions related to the PUM's state monitoring and event response decisions.

In other embodiments ML/AI models can be used to analyze and solve complex algorithmic problems in game theory, which can be applied, for example, to various scenarios including resource selection, pricing optimization strategies, and/or resource allocation. ML models can find optimal or close-to-optimal solutions and can predict possible outcomes in these scenarios. Probabilistic Graphical Models (PGMs), for example, is one of the techniques that can be used effectively for analysis of complex problems and interactions in these embodiments. PGMs is a class of statistical models that represent the probabilistic relationships between different variables through graphical representations. PGMs can be used to model the complex dependencies between agents and their strategies, providing a probabilistic framework for analyzing the possible outcomes of strategic interactions. This can be particularly useful for identifying malfunctioning sensors or other devices, based on large discrepancies between expected/predicted data and/or data patterns and actual data and/or data patterns generated by such devices.

In some embodiments a system that combines ML/AI and Game Theory (GT) includes a Data Collection and Preprocessing subsystem, responsible for collecting, cleaning, and preprocessing the data for further analysis, and may involve data pipelines, data ingestion mechanisms, and data preprocessing techniques. It can also include a ML/AI Model Training and Evaluation subsystem, dedicated to training the ML/AI models using appropriate algorithms and techniques, such as Deep Reinforcement Learning (DRL), involving the implementation of the chosen ML/AI models, the configuration of hyperparameters, and the evaluation of the model's performance based on specific metrics. For enabling the ML/AI model or models role in the games, they can be integrated with one or more Game Frameworks, which involves interfaces and mechanisms that allow the ML/AI models to interact with the game theory algorithms and decision-making processes effectively. Other subsystems, for example, can be used for real-time data processing and decision-making, for embodiments where real-time decision-making is crucial, such as in emergency event management scenarios, a dedicated module can be included to process incoming data, update the models' parameters, and generate timely responses or decisions based on the integrated ML/AI and game framework.

Data for ML/AI model training datasets can come from multiple sources, including historical and real-time data and/or data patterns from sensors and/or stakeholder interactions, simulated stakeholder interaction data and/or data patterns, simulated sensor data and/or data patterns, or a combination of two or more of these data sources. In some embodiments, the generation of realistic datasets for these simulated environments can also use ML/AI techniques, such as Generative Adversarial Networks (GANs). GANs are a type of unsupervised learning model consisting of two neural networks, a generator, and a discriminator, that are trained simultaneously. GANs can be used to generate synthetic data that mimics the behavior of agents in strategic interactions, thereby creating realistic simulated environments for decision and outcome analysis, and for ML model training purposes.

In some embodiments one or more ML/AI modules, using for example regression modeling, may evaluate the set of strategies that a set of players involved in a game may undertake. This can include identification of the optimum strategies for one or more players, for example a stakeholder, based on differing payoff parameters. In some embodiments this can include quantal response functions, where there is a mapping of expected payoffs based, at least in part, on possible mixed strategies. For example, if a player uses a strategy determined, at least in part, by an ML/AI module, this can result in that stakeholder optimizing their outcome. However, as the ML/AI module may identify alternative strategies for that stakeholder whereby the outcome, although not completely optimized for them, still represents a benefit and produces a benefit for other players, including other stakeholders, the stakeholder may opt to adopt, or may be directed to such a strategy as the benefits may be distributed across multiple stakeholders, including the PUM, with minimal or no adverse impact on any of the participants. In this manner the ML/AI modules may represent strategies that can incentivize all the stakeholders to operate in a cooperative manner whereby each stakeholder achieves benefit, without disadvantaging another stakeholder.

This can be particularly important in identifying the one or more incentives that a stakeholder has, which may be explicit or implicit, and as such influencing their behaviors in manner that, in the case of wellness, care and/or safety creates a state that is supportive of the PUM's overall condition. Such an approach can also be deployed in evaluating the near and long term impacts of the actions of one or more stakeholders, including the PUM, and as such one or more games may be dedicated to determining these impacts.

For example, those stakeholders that are focused on immediate benefit to themselves, for example a provider of a product and/or a service, may be inclined to optimize the price they receive, whereas there can be a longer-term benefit by balancing the single factor, for example cost, with a more comprehensive set of factors, such as time, repeat usage, impact on the PUM and the like.

The application of machine learning (ML) and more broadly AI (Artificial Intelligence) with game theory offers a number of potentially advantageous outcomes. The use of ML/AI to evaluate one or more data sets provided by one or more sensors, devices and/or systems can detect, inform and/or organize, in whole or in part, patterns of such data that may form part of one or more games. For example, there may be specific games that are focused on certain aspects of the data and/or the sensor, device and/or system operations, for example this can include, accuracy detection games, speed of detection games, resource cost games, fault or other performance characteristics games and/or the like.

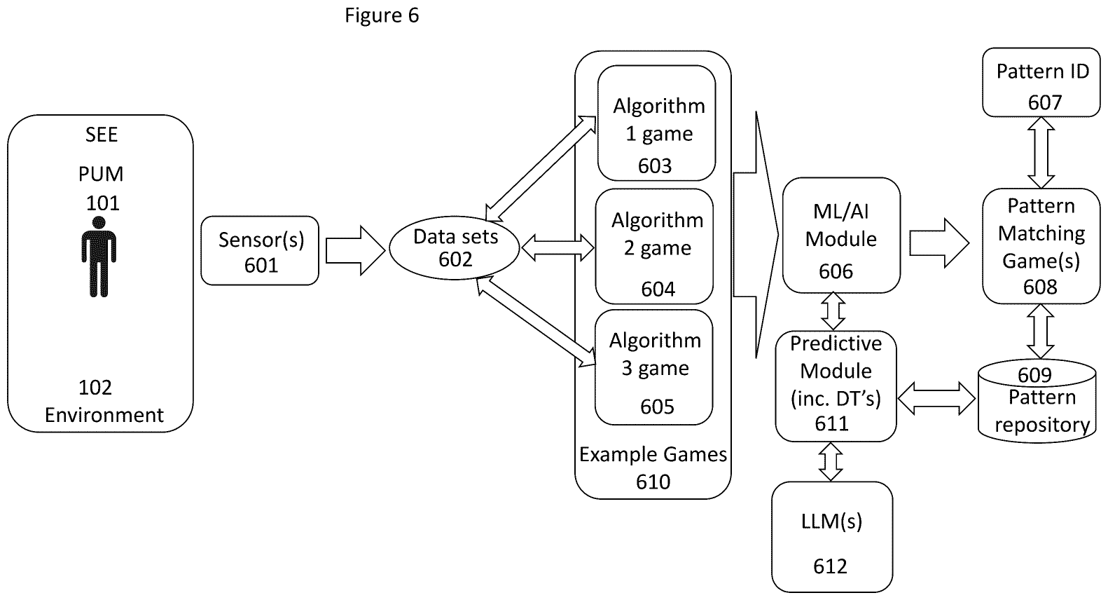

FIG. 6 illustrates an environment (102) with a PUM (101) being monitored by one or more sets of sensors (601), generating one or more data sets (602). These data sets (602) may form, as may the sensors (601), part of one or more games (610), for example those involving algorithms, for example Algorithm 1 (603), Algorithm 2 (604) and/or Algorithm 3 (605). The outputs of such games (603,604,605), may be communicated to one or more machine learning/AI module (606), the output of which may be communicated to one or more pattern matching games (608). One of the techniques employed by ML/AI module, either as part of a module or as an independent module, for example may include reinforcement learning models and may include, for example, predictive modules employing digital twins (611) and/or one or more LLM's (612). The pattern matching games may interact with pattern identification systems (607) and/or one or more pattern repository (609).

In this example, the ML/AI processes may act to identify those characteristics of the data sets generated by the sensors, devices and/or systems that represent the game parameters. For example, accuracy may be determined through evaluation of the data of one or more sensors over time referencing the data itself, data of other sensors that correlates to that data and potentially third-party data. For example, in the case of temperature, there may be multiple sensors capable of determining temperature as well as other environmental systems, such as HVAC. In another example, a particular metric may be known and used as a reference, for example a trusted or calibrated source of data, for example a reference time, temperature, humidity or other sensor measurable parameter may be employed to establish the source of truth, and the data sets of the one or more sensors over a time period compared to this reference to establish any degree of drift or loss of accuracy. In this manner the ML/AI may act to detect variation across a data set that indicate such a drift and/or loss of accuracy that is occurring and/or may occur at a future point.

These characteristics may then be used, at least in part, to form games comprising the one or more sensors as players whereby each of the sensors attempts to optimize the outcome of the game through, for example adjustment of that sensor's configuration and/or other sensor's configuration's.

A further deployment of ML/AI is to evaluate the outcomes of the one or more games that are being operated, with for example one or more sensors, devices and/or systems participating as players. Where each of the games is focused on a specific attribute or set thereof of the sensor's operations, for example, accuracy, speed and/or resource cost that is for example battery usage. In this example an ML/AI processing systems may operate on the outcomes of such games so as to identify one or more patterns of behaviors, indicated in part through the strategies deployed by each of the sensors and the outcomes of the games, to for example, provide data for a further game, for example a balance game, which is designed to establish the optimum balance between the potentially competing outcomes of each of the individual games.

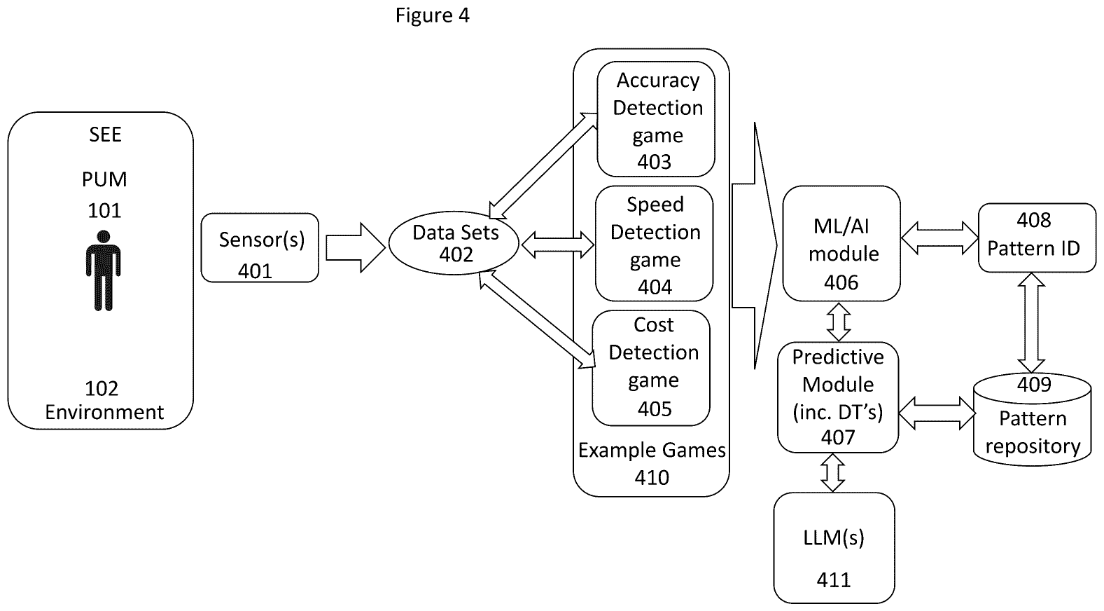

FIG. 4 illustrates an environment (102) with a PUM (101) that is monitored by one or more sensor sets (401) generating one or more data sets (402). In this example, such sensors set (401) and data sets (402) can form part of one or more games (410), each of which has strategies, outcomes and/or payoffs that represents a particular aspect of the sensors operations and resultant data set. For example, Accuracy detection game (403), may be used to evaluate the accuracy of the data generated by such a sensor, and may include differing configurations of that sensor as part of the strategies of the game. This may be the case, for example, for other games, that for example can include, for example, speed detection game (404), cost detection game (405). In some embodiments, these games outputs and/or their inputs and/or strategies may be communicated to one or more Machine Learning/AI modules (406), which may invoke one or more predictive modules that include one or more digital twins (407). These games, (403/404/405), can generate a corpus that can be used by the ML/AI modules (406) and/or predictive modules (407) and/or one or more LLM's (411). The outputs from the machine learning/AI (406), predictive modules (407) and/or LLM (411) can be communicated to one or more pattern identification modules (408) which may interact with one or more pattern repositories (409). This can include identification of patterns that are derivative of existing patterns.

Such an approach may also provide data sets to one or more predictive processing modules, including for example digital twins, so as to anticipate future operations and outcomes of the games. This can include the ML/AI processing undertaking the role of one or more player in one or more games to establish one or more strategy for those games. This may result in an ML/AI player that operates to establish the optimum strategy for the game and as such may form a reference to which the operating sensors, their strategies and the data there from may be compared. This can be particularly useful in determining whether such a sensor has or is about to enter one or more failure mode or other detrimental operating configuration and/or processing and the like.

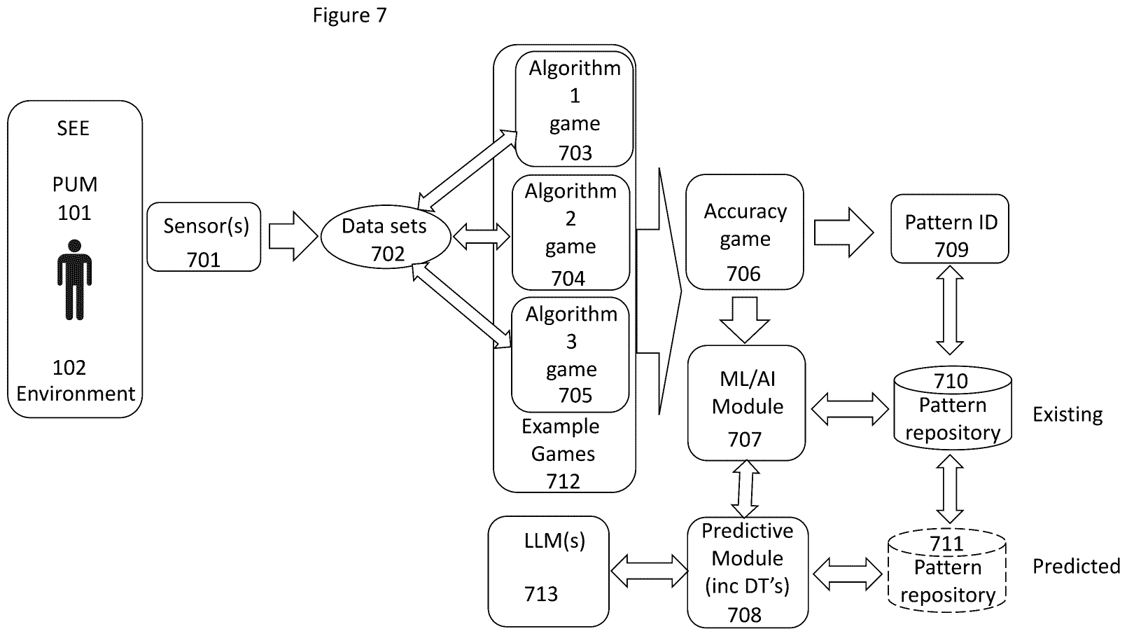

FIG. 7 illustrates an example embodiment (102) where a PUM (101) is monitored by one or more sensors sets (701), generating one or more data sets (702). In this example embodiment, such data sets (702) and/or sensor sets (701) form part of a set of games employing differing algorithms, for example algorithm 1 game (703), algorithm 2 game (704) and/or algorithm 3 game (705). For example, for such an algorithm may include reinforcement learning, weightings, deep learning, regression and the like. These games outputs, payoffs, inputs and/or strategies may be, for example communicated to one or more further game, for example an accuracy game (706), where such game may interact with one or more machine learning module/AI (707) and/or one or more predictive modules, including one or more digital twins (708) and/or one or more LLM's (713). These games (706) and modules (707/708/713) may interact with one or more pattern identification modules (709) and/or pattern repositories (710). For example, where there is a new predicted pattern, this may be stored in a pattern repository (711) for such predicted patterns.

In some embodiments, one or more optimization games based on state, patterns and/or behaviors can be employed to obtain optimum result set for evaluating PUM state, for example, through taking an initial measurement and then undertaking subsequent measurements, using for example a differing combination of sensors, representing different strategies. For example, a change in pattern, behavior and/or state based on at least one sensor data set, where game is employed for detection of such changes and ML/AI acts to optimize sensors for such games.

A further deployment of ML/AI processing may be in the form of determining, in part or in whole, the one or more algorithms that can be deployed on the data sets of the one or more sensors. This may include the ML/AI processing using such techniques as reinforcement learning to identify, at least in part, algorithms that are capable of producing data sets that represent the characteristics of the sensor sets in aggregate. For example, this can include development and deployment of algorithms that may incorporate the data sets of a set of sensors, such that each of the sensors data sets is evaluated in manner that produces a consistent and cohesive outcome, for example in the form of metrics and/or other representations. Such algorithms may include one or more weightings, priority, probability and/or other variables that can be deployed on the sensors data sets. In some embodiments, such weightings may form part of an attention mechanism for one or more LLM.

One aspect of the deployment of ML/AI processing is the identification, confirmation, validation and/or verification of the state of an environment using the data sets generated by one or more sensors, devices and/or systems monitoring that environment. In some embodiments the state of an environment and the PUM therein can be represented by one or more patterns and/or behaviors, which can be represented by tokens. Although these quantized states can represent patterns and/or behaviors of the PUM within an environment, there may be edge and/or transition states that represent a change in these states, for example from a quiescent state to an event state. The ML/AI processing may be used, in some embodiments with one or more games, to identify these edge and/or transition states, potentially in advance of those states occurring. This can include the use of digital twins where, for example, multiple games may be employed to, at least in part, predict which of the possible states is most likely, based on the flow of data from the one or more sensors, devices and/or systems. For example, there may be a sets of data generated at T1 (time1), which could indicate a number of possible state changes, including a null state change, however such data may be outside one or more configuration thresholds and as a consequence may be passed to the one or more ML/AI modules for evaluation, including the use of one or more digital twins. As further data is generated, for example at further time periods (T1, T2 . . . Tn), such data may be passed to one or more games, where this data, for example as quantized tokens, forms the moves in those games. As these moves unfold, the determination of the most likely state change can be predicted, where for example the outcomes of the games represent the potential patterns and/or behaviors of the PUM and the set of potential event states. In this manner the ML/AI processing may, for example, employ one or more games that are designed to represent state changes, where the ML/AI, for example in form of an AI module may act as a player within such games. Such games can include multi-level reasoning (level-k reasoning), Bayesian games, multi-stage games, hierarchical games and/or the like.

In some embodiments, games can be used as indicators of transition states, whereby a PUM is transitioning from one HCP state to another, multiple games may be invoked so as to determine the most likely potential transitions, and prediction of the set possible alternate, and potentially new, states. In some embodiments, one aspect of the state change is the evaluation of the data sets, to determine that the data represents an actual change of state. One of the many challenges for effective multi sensor monitoring of a PUM in a SEE, is the ability to determine with a high degree of accuracy and reliability the one or more state changes in that environment and the accurate and timely determination of those changes transformed into events and/or actions that can have a positive benefit on the wellness, health and/or safety of the PUM.

In some embodiments, games, such as those employed in the volunteers dilemma can be employed, where the determination of whether the data sets representing the state of the SEE and the PUM therein are consistent with a change of state that can be represented by one or more event states, that can include one or more actions, where these actions have an impact on the PUM. These games can involve multiple sensors, devices and/or systems providing data sets, that can, at least in part, represent a change in the state of the SEE. As the number of sensors, devices and system can be represented by N, each of which can be a player in the game, the use of symmetrical games where there are mixed strategies, can involve multiple (N) pure strategy equilibria. In this manner if a sensor, device and/or system generates a data set that represents an event, as determined by the configuration of that first sensor, device and/or system, and one or more other sensors, devices and/or systems contemporaneously generate data sets that do not represent that event, then in the game that involves the first sensor, device and/or system as a player and the other sensors, devices and/or systems also as players, the probability of that first sensor set event is an actual event declines with the number of other sensors, devices and/or systems contributing data sets.

However, as these data sets are generated over a period of the time, if in subsequent time periods the other sensors, devices and/or system generate data sets that represent a state change and event state, the probability of an event occurring increases. The determination of which event type can be undertaken by one or more care hub and/or care processing systems, which can, at least in part reference one or more repositories of events.

In some embodiments, AI/ML modules may be configured to optimize one or more sets of sensors, devices and/or system to reduce probabilities for those sensors and the data sets they generate to a minimum set of alternatives that represent the state of the PUM, environment and/or other stakeholders.

### State and Games

On aspect of the system is the monitoring of the state of the PUM and their environment, such as a SEE. In some embodiments there can be games that have strategies and operations intended to identify, at least in part, changes in the state of a PUM in an environment. For example, if a PUM is undertaking their daily routine, and the patterns and/or behaviors being monitored conform to their time of day (ToD) behaviors, expressed as tokens, and the respective quiescent behaviors represented by bevokens, one or more transition games may be operating to determine the potential that a PUM behavior and state is shifting. This can include games that operate over extended periods, for example months, whereby although the data sets represented by the tokens are within the thresholds of those tokens, the small deviations within those data sets can be represented by variations in the strategies employed by the one or more sensors acting as players in those games. For example, if a sensor data set includes increasing or decreasing values toward a threshold and/or exhibits increased volatility in the data sets, this can impact the strategy of the sensors in, for example the game of accuracy, timeliness and/or resource cost that the sensor may be undertaking. In some embodiments one or more ML/AI modules may be employed to, for example, generate a set of vectors representing such variations.

Such minor variations may be monitored by a second set of games, the transition games, where each of these contextually minor variations forms part of one or more games intended to identify the probability that the care, wellness and or safety state of the PUM is changing.

In some embodiments, this will involve the deployment of multiple transition games, for example each of which is embodied in a digital twin of the PUM and their environment, which can include, for example the use of a PPE (Personal Physics Engine). Such a set of games may then produce a set of outcomes that are evaluated by one or more ML/AI modules, which may also be applied to the one or more games to determine best fit strategies for those games.

There may also be a hierarchical structure such that all the games being operated within these digital twins are aggregated to a further one or more games to determine the collective outcomes of those games. In this manner the various potentialities expressed by each of the games within each digital twin can be evaluated for an overall likelihood that there is one or more state changes occurring with the PUM and their environment.

### Games Representing State

The use of tokens as representations of behaviors, data sets and/or communications enables one or more games to operate on and/or with those tokens. In this manner players in the game may deploy strategies based, at least in part, on these tokenized patterns and/or behaviors. For example, a sensor, device and/or system may be playing a sequence of event games, where the data of the sensor is played against a second player represented by a data set that represents the thresholds of that data. That is the thresholds form the boundaries of the data sets, which if exceeded, can represent an event. For example, if a visual sensor is capturing the edges of an image, for example a person in an environment, and those edges move at a rate that exceeds the normal range of motions for that person in that environment, for example as represented by a personal physics engine (PPE), then this could be expressed as exceeding the thresholds and as such could be an event. In terms of the game, each sequence of play could be represented by two players, the sensor data as captured in real time and expressed as a token, and the threshold data expressed as a second token. Each token includes a value, which for the threshold token is fixed by the configuration data of the sensor, device and/or system, whereas the data of the sensor, device and/or system representing the current state of the PUM and their environment is dynamic, based at least in part, on the data collected by that sensor, device and/or system. In this manner each token value determines the payoff for the game, in that if the value of the real time data token is higher that the threshold, then an event token is the payoff.

In some embodiments, such a token may then be passed to one or more other sensors, devices and/or systems for further evaluation, action and/or processing. This can include such a token being used in further games. One aspect of this approach is as each game is played there can be a certain time period in which the sensor data set is collected and transformed into a token, which can be aligned to the one or more patterns and/or behaviors of a PUM. For example, if a PUM is sleeping the time period may be longer than if they are moving, exercising, cooking or otherwise being active it may be shorter.

The determination of the state of the PUM through the playing of a sequence of games with one or more sensors, devices and/or system supports the privacy of the PUM in that if the state is quiescent, the tokens may be retained for a period and then deleted, with only the sequence of the state being retained. The period of retention may be determined, at least in part, by the activity of the PUM and the sequence of patterns and/or behaviors of that PUM. This may be evaluated, for example, by a risk module.

In some embodiments, the use of tokens for data transfer embodies a quantization of those data sets, such that each sensor has normalized the data in conformance with that sensor's configuration, including for example one or more thresholds, and as such the state of a PUM in an environment may be represented by sets of such tokens. This may include tokens which to a greater or lesser degree have conflicting state information, for example a sensor has issued a token indicating an event and another sensor in the same environment within the same time period issues a token indicating a quiescent state. In this example one or more care processing systems may reconcile these conflicting state representations through, for example, deployment and/or configuration of other sensors, devices and/or systems in such an environment. For example, a camera may be configured to provide visual images in addition to edge detection data, enabling a more accurate representation of the state.

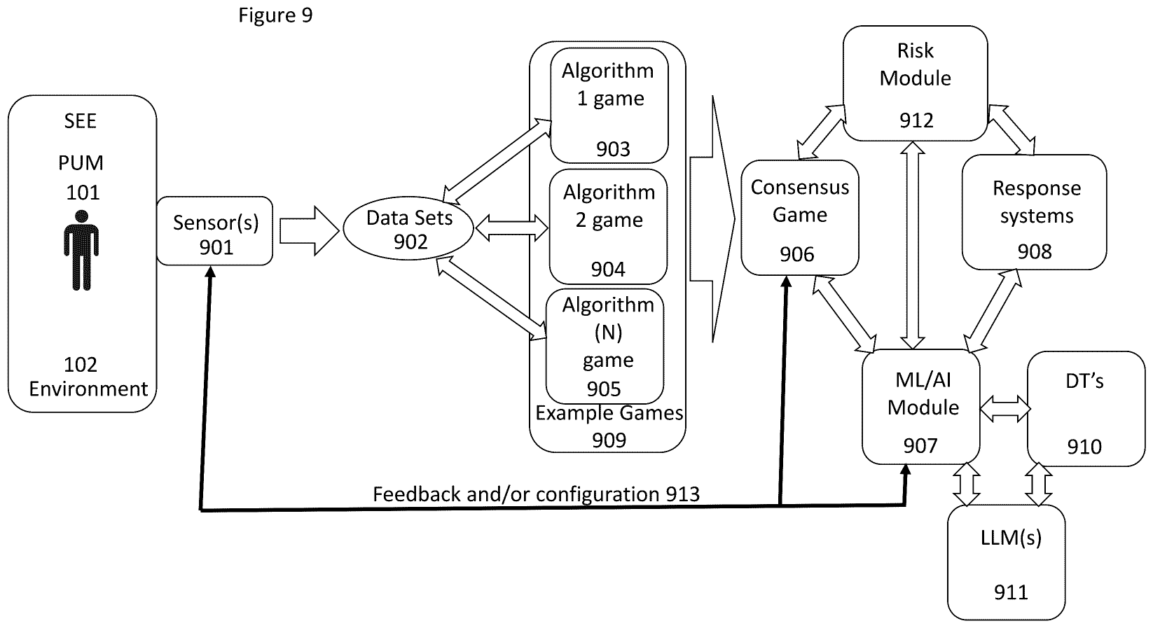

FIG. 9 illustrates an example embodiment (102) where a PUM (101) is monitored by a set of sensors (901) which generate one or more data sets (902). These data sets form part of one or more games (909) employing one or more algorithms, for example algorithm 1 game (903), algorithm 2 game (904) and algorithm n game (905). For example, the outputs and payoffs of these games, may provide contradictory, orthogonal, disparate, or other outcomes that have at least a degree of variance that is incompatible with a common agreed conclusion. For example, this can include outcomes that exceed one or more thresholds, have insufficient accuracy, exclude or include other deviations and the like. In this example one or more consensus games (906) may operate, for example in combination with one or more machine learning modules/AI (907), which may in turn invoke one or more digital twins (910) and/or one or more LLM's (911), to ascertain an outcome that represents the consensus of these divergent outcomes. This consensus may then be communicated to, for example, one or more risk modules (912) which may communicate with one or more response systems (908). In some embodiments, consensus game (906) and/or machine learning module/AI (907), digital twins (910) and/or LLM's (911) may communicate with sensors though a feedback and/or configuration systems (913) so as to achieve and/or further refine the consensus outcomes.

In some embodiments, this approach supports a deterministic and/or probabilistic consistent evaluation of the state of a PUM in an environment, including for example, through one or more statistical, machine learning and/or AI techniques, where the quantization enabled through the use of tokens provides an accurate representation of that state. For example, a set of inputs, for example those to one or more games, represented as a set of tokens, can produce a consistent output of such games. In this manner the state can be represented by one or more games, where the inputs, the tokens and the outputs, which may also be tokens, are consistent. This supports an efficient and accurate approach to establishing the care, wellness, health and/or safety condition of a PUM in an environment through the identification of the state of that PUM represented by tokens that represent their patterns and/or behaviors.

In some embodiments, a directed graph may be used to represent a game. This can comprise a set of decision nodes which can be associated with a sensor, device and/or system acting as a player in the game and the tokens generated by such sensors, devices and/or systems that can be deployed within such games

In some embodiments, games may be embodied that involve the communication of information, where there is a sensor and a receiver as players of the game. For example, this could include one or more sensors, devices and/or systems. In such an example the first player may have a set of information, for example a data set represented by a token which is by its nature private. This token is a message sent by the first player, that is as an action in the game, where a second player observes the message and undertakes a second action.

These type of games are known in the art as “cheap-talk” or information transmission games. One aspect of this approach is that there may be an inherent conflict of interest between the players in that for example, the behaviors monitored by one player, for example one or more sensors, devices and/or systems are configured and operated to monitor the quiescent state of a PUM, whereas the second player, which can be one or more sensors, device and/or system is configured and operated to detect one or more events impacting the wellness, safety and/or care of the PUM.

In some embodiments there may be a set one or more sensors, devices and/or systems that are monitoring a PUM in an environment that generates one or more data sets. This data can represent patterns and/or behaviors, including sequences thereof, and can be represented as tokens. In this example this can represent the available common knowledge domain (K) of the monitoring of the PUM and their environment. In this example the first player, representing a first sensor, in sensing the common knowledge domain (K) of the PUM under monitoring may undertake some action (a), for example a token representing a data set, where that token in a subset of the possible tokens that can be generated by such a first sensor (A). The knowledge domain, may for example comprise the quantized monitoring states of the PUM expressed as patterns and/or behaviors represented by tokens. A second player may observe this action (a) and respond with an action, for example this may be a token representing a configuration of the first sensor and/or configuration of one or more other sensors, devices and/or including the second sensor (b), where such action is a subset of the available actions (B). In this example the payoffs may be S1 (b,K) and s2 (b,K).

In some embodiments, one sensor can request data from another sensor, for example, if sensor (A) captures audio and sensor (B) captures impact (haptic), then for example, sensor (A) games payoffs can include sensor B data, for example where that data is expressed in relation to, for example a threshold.

In some embodiments a sensor may be configured with one or more event types that, based at least in part, on the capabilities of the sensor, the configuration of the sensor and the appropriate thresholds, which in some embodiments can be represented as quantized events, represented as tokens.

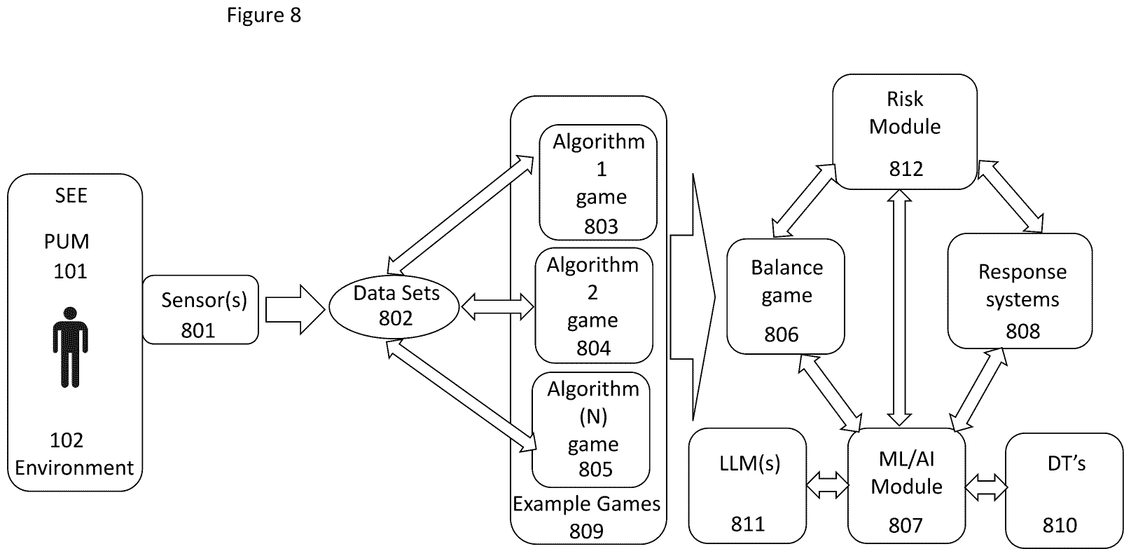

FIG. 8 illustrates an environment (102) where a PUM (101) is monitored by one or more sets of sensors (801), which generate one or more data sets (802). These data sets form part of one or more example games (809) employing one or more algorithms, for example algorithm 1 game (803), algorithm2 game (804) and algorithm n game (805). The outputs of these games may offer differing perspectives on the state of the PUM in the environment, where for example one game, for example algorithm 1 game (803) may provide an output where the state of the PUM (101) in the environment (102) is quiescent. Algorithm 2 game may also provide this output, whereas a further game, for example algorithm 3 game (805) may provide an output that is an event. In this example there may be a balance game (806) that establishes the balance between the multiple games and their outputs. This balance game (806) may integrate with one or more machine learning/AI module (807), where for example the one game that has generated an outlier, that is an event rather than quiescent, is invoked to, for example using digital twins (810), undertake predictive processing, including using for example one or more LLM (811) on that outlier. The balance game (806) outputs may be communicated to one or more risk systems (812) for communication with one or more response systems (808) as may the machine learning/AI module (807) outputs.

One aspect of deploying games on sensor data, where for example the sensor is a player of the game, is detection, at least in part, of an event at the earliest possible stage and with the most accuracy. For example, a sensor, device and/or system, may be involved in two games, one which has a quiescent state versus an event state as the possible outcomes and another operating in parallel that has outcomes and payoffs configured for accuracy of the data set, for example in matching event or other states. In some embodiments, the outputs of these two games may be passed as inputs to a further game, for example a balance game, where the outcome of that game is the determination of the relative values for these potentially contradictory states. For example, if the event game has an event outcome with a high value and the accuracy game has a high accuracy value, then the balance game output is an event with high accuracy. However, there may be further considerations, such as resource use, for example battery consumption, privacy of the one or more stakeholders' being monitored and the like, which in some embodiments may be represented in further games and/or as players in some games. This can include the use of one or more digital twins, which in whole or in part, may deploy the operating games with the sensor generated data sets so as to evaluate the potential strategies and outcomes of such games. These outcomes may be incorporated in any responses, for example those enacted by one or more response systems.

Such multiple games and the data sets thereof may be used, in whole or in part by one or more ML/AI module for training such module so as to create one or more models of possible game strategies, outcomes and/or payoffs.

In some embodiments the outputs of the one or more games may be evaluated by, for example, one or more algorithms, such as consensus algorithms, byzantine algorithms and the like.

In some embodiments, games may involve players, in the form of stakeholders, that are either human or machine such as sensor, device and/or system, which can include one or more AI/ML modules. This can include hardware and/or software that acts as a proxy for a human stakeholder. For example, this may include an ML/AI module that is configured to assist a stakeholder in playing one or more games, where for example such a module may provide strategies and/or other assistance to a player. For example, this can include direct intervention in the game play and/or provision of prompts or other interfaces on devices that are controlled by the PUM or another stakeholder. This can include the provision of such assistance in the form of text, images, audio, haptic and/or other human interpretable methods.

In some embodiments this can include AI/ML modules that act as players in one or more games, where such modules are configured, potentially by one or more stakeholders, including sensors, devices and/or systems to represent those entities.

In some embodiments, one consideration is the incentives, attitudes and/or intentions of the human stakeholders. This is particularly the case with those human stakeholders who are involved and/or invested in the care, health, well-being and/or safety of a PUM.

This includes those stakeholders who are professionally engaged in the care and/or well being of the PUM, for example, carers, medical professionals and the like and those who engaged through family ties and/or friendship. Those professionally engaged generally operate within the agreed terms of their engagement, such as contracts, service agreements and/or the like, where the care village systems such as audit modules and performance metrics, including those involving games, can be used to monitor and support the wellness and care of the PUM.

However, those stakeholders that do not have such agreements and/or arrangements, such as family and friends, where the social interactions are determinative of their behaviors in relation to the PUM. For example, if a friend or family member likes or dislikes a carer may impact the carer behavior in relation to the PUM. In some embodiments this can, at least in part, be mitigated by the use of one of more games where the interactions of these stakeholder is in part influenced by such games. For example, if the stakeholder trusts the carer, this may be represented in the form of a game payoff, where the carer behaviors determine the outcome of the game, represented by one or more tokens. In some embodiments, games that represent social dilemma may be deployed.

The use of games as an intermediatory for the dissemination of data regarding the PUM, where such data is represented by one or more tokens, enables the care village systems to provide differing potential outcomes for those stakeholders and/or the PUM. For example, reputation data may be presented in the form of a game, where the carer operates to achieve the maximum reputation rating in regard of their behaviors in regard of a PUM. This reputation data may then be communicated to other stakeholders, such as a second PUM, their friends and family, such that the reputation, as presented to the second group may include data from the friends and family of the first PUM, all of which is provided in a manner that protects the privacy of the first and second groups.

In some embodiments, there can be sequences of games, where for example, some game outcomes can provide input to further games in a sequential, hierarchical, taxonomical, ontological, and/or other arrangement. This can include games played in parallel and/or in sequence in any arrangement.

In some embodiments, a comparison of potential outcomes of the one or more games being undertaken in a sequence and/or in parallel, can form part of the determination of responses, actions and/or events.

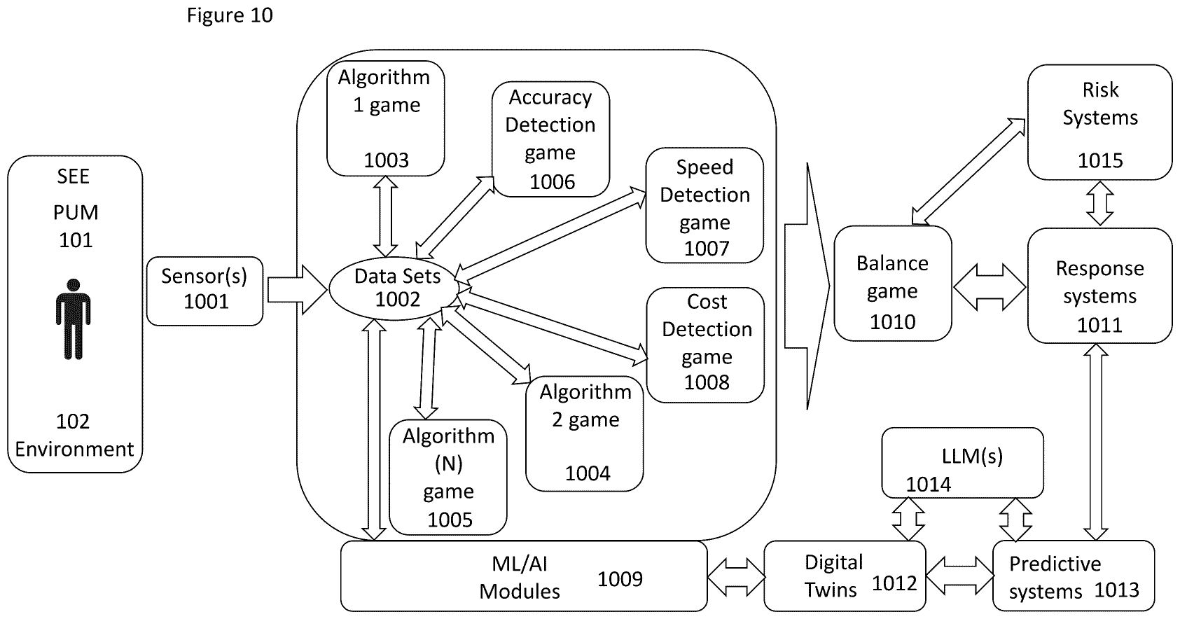

FIG. 10 illustrates an environment (102) where a PUM (101) is monitored by a set off sensors (1001), which generate sets of data (1002). These data sets (1002) may then form part of one or more games, for example Algorithm1 game (1003), Accuracy detection game (1006), speed detection game (1007), cost detection game (1008), Algorithm 2 game (1004) and/or algorithm (n) game (1005), in any arrangement. These games may then have their outputs, strategies and/or payoffs communicated, in whole or in part in any arrangement to one or more further game, for example a balance game (1010). Such a balance game (1010) may then communicate with one or more risk systems (1015) and/or response systems (1011). The data sets (1002) may also be communicated to one or more machine learning/AI modules (1009), which may include AI functionality, such as for example generative AI, deep learning, neural networks and the like. These modules (1009) may then employ one or more digital twins (1012) and/or one or more LLM's (1014) and/or one or more predictive systems (1013), which may communicate with one or more response systems (1011). Such response systems (1011) may communicate with one or more risk systems (1015) to, at least in part, determine the appropriate response output.

In some embodiments, some games may be played repetitiously on a time line, with each outcome forming part of a data set, for example in one or more digital twins. In this manner sets of games may represent a series of patterns and/or behaviors and/or interactions of one or more stakeholders over a period of time, which may be contiguous or asynchronous. Games deployed within one or more digital twins can represent one or more transition states, including predictive states.

In some embodiments various types of graphs, such as acyclic directed graphs (ADG) may be used to represent the outcomes of the sensor, device and/or system operated games and/or the digital twin operated games.

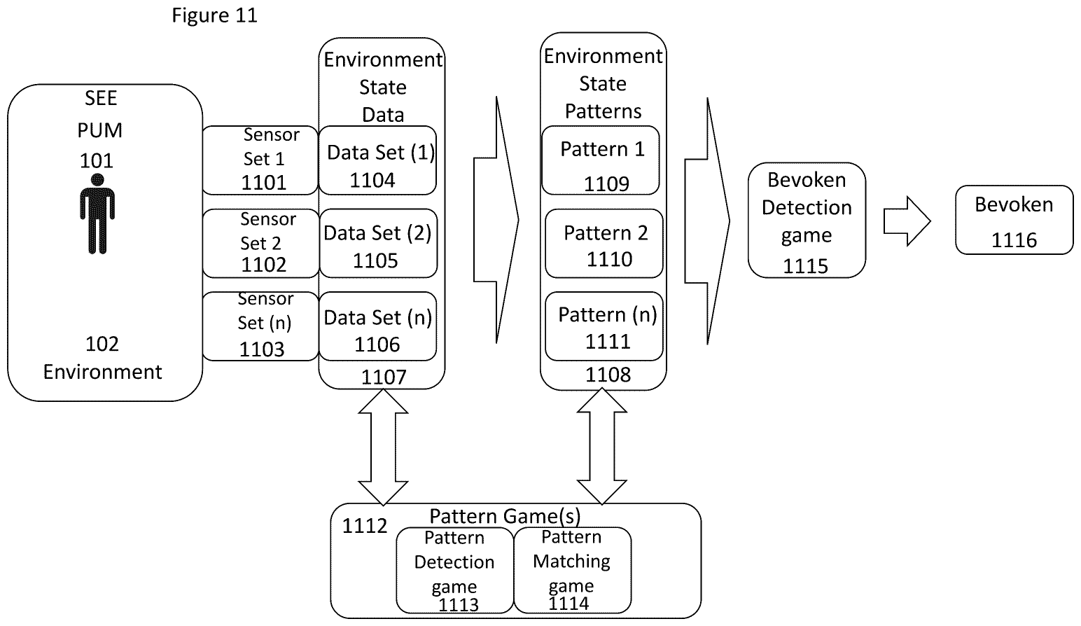

FIG. 11 illustrates an example embodiment (102) where a PUM (101) is monitored by a set of sensors, for example sensor set 1 (1101), sensor set 2 (1102) and/or sensor set (n) (1103. These sensor sets generate data sets, for example sensor set 1 (1101) generates data set 1 (1104), sensor set 2 generates data set 2 (1105) and sensors set (n) generates data set (n) (1106). These data sets in aggregate represent, in part and/or in whole, the environment state data (1107). Such data, either from the individual data sets (1104,1105,116) and or in aggregate (1107), may be evaluated in any arrangement by one or more pattern detection games (1113) comprising part of a set of pattern games (1112). Such detections may be matched through one or more pattern matching games (1114) so as to create, at least in part one or more environment state patterns (1108). For example, each of the sensors and data sets (1101,1102, 1103) and (1104,1105,1106) respectively may through pattern games (1112), generate and/or match a set of patterns for example pattern 1 (1109), pattern 2 (1110) and pattern (n) (1111). These patterns may be communicated to one or more bevoken detection games (1115), which in turn may generate one or more bevokens (1116) representing such detected bevokens.

For example, a payoff may be higher in, for example, a sensor game where the sensor wins when the state is not quiescent, for example, when an event is detected. This can include situations when a transition from one state to another is taking place, for example a PUM is transitioning from, for example, watching TV to sleeping. In this example sleeping is initially an event, and the sensor may win a game by recognizing such, though subsequently, the sleep state can be represented by a Bevoken, the sleep Bevoken, which can have a quiescent state until another transition occurs.

In many situations involving a PUM and their care, wellness, health and/or safety the behaviors exhibited by the PUM and data generated by the sensors, devices and/or systems monitoring the PUM and their environment may be subject to subjective interpretations by one or more stakeholders. For example, family and/or friends may see a change in behaviors of a PUM in a more negative or positive interpretation than the monitoring data represents. In these situations, the tendency may be for such stakeholders to over or under react to the situation, which can have a detrimental impact on the PUM.

One aspect of the care village may be the provision of such data regarding the behavior of the PUM in the form of a game to the other stakeholders, such that they may determine the most appropriate response to the changing behaviors of the PUM. For example, the stakeholder may be presented the behavior change in the form of a communication to an application that resides on their one or more devices, where the state of the PUM is represented as a set of choices representing the possible responses of that stakeholder.

This can be in the form of a game, however, the presentation of the data and choices may be in the form of tokens representing the behavior states, and as such may protect the privacy of the PUM. These tokens may then be arranged in a manner such that the stakeholder can evaluate which of the potential responses to the situation will have an impact on the PUM. The stakeholder may play such a game for their own benefit, rather that the care and wellness of the PUM.

In some cases the responses of the stakeholder may be based on their beliefs, which may, in whole or in part, not match the data sets generated by the sensors, devices and/or systems monitoring the PUM. In some embodiments this can result in such a stakeholder determining a response or choice of action that is not necessarily in the best wellness, care and/or safety interests of the PUM. For example, this can include actions that may impact the longer term care and wellness prospects of the PUM.

In some situations, there may be an ML/AI player that is part of the game being undertaken by the stakeholder as they consider their responses to the change in state of the PUM. Such an ML/AI module may be configured to act to the benefit of the PUM. In other situations, such an ML/AI module may be configured to act to the benefit of a stakeholder who is not the PUM. There can be games where there are multiple ML/AI modules with differing configurations operating on behalf or and/or assisting one or more stakeholders.

For example, such an ML/AI player may employ strategies that present alternative outcomes. For example, if the game payoffs include financial data, such as the cost of additional care a PUM may need to address their changed state and circumstances, the AI/ML module may adopt a strategy that shows the long-term costs versus the short-term costs so as to illustrate, for example, that a set of actions may cost more in the short term but cost less over the longer term. This approach may support mitigation of the stakeholders potential actions or responses through the provision of additional alternative possible actions and/or responses, and as such influence the adoption of such strategies within the game that present outcomes that although they do not optimize the immediate interests of the stakeholder, create incentives for that stakeholder that do not materially disadvantage the stakeholder and that are beneficial to the PUM or mitigate any detrimental impact to the PUM.

One aspect of such a situation may be the provision to the PUM of additional resources that support the PUM's care and wellness as their health state changes, for example further medications, care visits, changed environment and the like. In this example one or more other stakeholders, such as family, insurance provider, care provider, health professional and/or other stakeholders may all have incentives to create differing outcomes for the PUM.

This can include financial, health, emotional, beliefs, availability, timeliness and/or other incentives which may impact the decisions, actions and/or responses of those parties.

For example, one set of games, can include wellness and care optimization games, which can in whole or in part be based upon and/or include one or more care, wellness, health and/or safety metrics, configured to ensure, from the perspective of the PUM, that their wellness is maintained and/or improved. A second stakeholder, for example a medical professional may also want the PUM wellness and care is maintained and/or improved, however their incentives may differ from those of the PUM. The same is the case for other stakeholders, such as carers, family, friends, other care providers and the like.

The use of game theory to accept these differing perspectives and through each of the players representing these stakeholders creates a set of actions, events and/or outcomes in support of these differing perspectives enables a fundamentally more responsive system for the PUM and stakeholders. Such an approach does not have the limitation of many current systems which are designed using use cases, where the unanticipated use case can cause potentially significate detrimental impacts for the PUM and potentially other stakeholders.

The use of tokens in games as the means of instantiation of the players strategies enables those games to represent the sensed behaviors of a PUM in environment without providing any personal data such sensing may observe. This can also apply to one or more other stakeholders interacting with such a PUM in an environment. This use of tokenization, in that game players only have access to tokens as representations of states and/or state changes without access to underlying data-support the privacy of those stakeholders.

In some embodiments, games may be arranged in manner to support early detection of one or more events. For example, this can include simultaneous and sequential arrangement of games. In some embodiments, payoffs for one game may be correlated to identification of specific behaviors represented by, for example, one or more Bevokens. This can include the use of one or more digital twins, where one or more games and the payoffs thereof may be operated to determine, at least in part, the likely outcomes of a situation under monitoring. In some embodiment an AI/ML module may operate as a player in such games to explore the potential strategies that may be employed by the one or more stakeholders' involved. In this manner the most likely stakeholder strategies, which in some embodiments may be indicative of such stakeholder incentives can be ascertained.

In some embodiments, there can be one or more matching systems, for example a matching module, where for example, combinations of tokens which represent one or more states, for example those represented by a set of patterns which may form one or more tokens, such as event or occurrence. In this manner such an event or occurrence may be identified and matched to, for example, one or more Bevokens or other representations, such that the underlying data, for example involving the privacy of the PUM or other stakeholders, is not revealed.

### Games and Game Types

In some embodiments a set of games involving game theory may be deployed within the care village. This can include one or more games based on tokenized data. One aspect of this deployment is the use of such games with sensors, devices and/or systems to identify the state of a PUM and/or other stakeholders in an environment, such as a SEE, including their interactions. These games can include all the main types of games that involve, at least in part, game theory, such as for example, cooperative and non-cooperative bargaining, normal form games, matrix representation games, extensive form games, multistage games, repeated games, Bayesian games, auction games, bidding games, signaling games, reputation games, information games and/or the like. This can include derivations of such games that correspond to particular behaviors of and among stakeholders, including those identified by one or more machine learning techniques instantiated, for example in an AI/ML module.

These games can involve various forms of equilibrium, including Nash equilibrium and variations thereof. Various examples are described herein.

### Multi-Stage Games

In some embodiments, sensors, devices, systems and/or stakeholders may be players in sets of games, for example a series of games, that are, for example, arranged as a hierarchy, tree, sequence or other arrangement. In this manner a player may adopt a strategy for each of the individual games. For example, a sensor may simply attempt to optimize the payoff in a single game, for example a game for event detection. This may be the case as the processing power of the sensor is limited and as such only capable of undertaking a single game at any one time. However, a more sophisticated entity, such as a device incorporating multiple sensors and greater processing, memory and other resources, may be able to undertake multiple games simultaneously, for example if the device has multiple sensors, then the device with sufficient processing power may be able to operate a game for each of the sensors thereof. Depending on the available processing power, the device may also be able to operate one or more games that aggregate and/or integrate the sensor games and their outcomes.

In the example of a system with sufficient processing power, such a system may be able to operate multiple games and consequently adopt a set of strategies that link across multiple games. A key aspect of this approach is the information available to such a system for each of the games being undertaken. For example, if the first game has an outcome of four possibilities and the next game is sequential then those four outcomes are the information set available to the system. However, if there are multiple games being played simultaneously, for example, where a sensor with limited processing is undertaking a single game, for example, an event game, then the information set for the system is incomplete, though the system can have knowledge of the potential states of the outcomes of such games through configuration of that system, although the system will not know the values of such outcomes.

For example, if a set of sensors is playing a set of games to determine the state of a PUM in an environment, and these games can have an event or quiescent outcome, which can be represented by an appropriate Bevoken, the system may invoke one or more digital twins to explore the possible outcomes of such a set of games. In this manner the system may develop a set of strategies, for example using an ML/AI module to determine the most likely potential outcomes and/or the optimum strategy for determining the most accurate, efficient and timely representation of the PUM in the environment, even with contradictory sensor game outcomes.

A further aspect is the relationship between the equilibrium of each of the games in a multi stage game, such as one being played by a system with sufficient processing power, for example a care hub and/or care processing system Each of the individual, sub games forming the multistage game can be played by each sensor based on the data sets, which can be represented by tokens, in a specific time period. The outcomes of these games can be independent of the previous or future games played by such sensor. In this manner if the individual games played by a set of sensors have one or more Nash equilibria then the multi stage game, played for example by a system then has a unique subgame-perfect equilibrium.

### Repeated Games

Repeated games are those where stage games, that is independent games which although complete have imperfect information, for example can be those games deployed on a sensor, device and/or system.

As many of the sensors, devices and/or systems can play the same game multiple times over an extended period, there becomes a series of equilibria that represent, at least in part, the state of environment as detected by the one or more sensors. For example, this may represent the quiescent state of the behaviors, represented, for example, by Bevokens. In some embodiments, the time may be segmented on, for example, clock time and/or ToD token time to align, at least in part, the one or more states detected by the one or more sensors, devices and/or systems with such time metrics. This can provide a set of equilibria that can be used, at least in part, to train one or more ML/AI modules, including for example an LLM, to create a model representing the state of the PUM over one or more time periods.

In some embodiments an ML/AI module may operate as a player, for example in one or more games with one or more sensors, devices and/or systems. In this example such sensor, device and/or system may be providing tokens which represent a low degree of certainty as to the measurements of that sensor, device and/or system. As the game is repeated the AI may act to play a token in the game that provides a payoff that has a negative outcome for both players. In this manner a sensor, device and/or system where the measurements are of low certainty may, for example have over time a metric associated with the operative effectiveness of such a sensors, device and/or system decay. This decay may then result in such a sensor, device and/or system being replaced and/or repaired and/or having the data generated by such deprecated in any evaluation of such data.

### Transition Games

In some embodiments there may be games that are designed to identify, at least in part, transitions between one state of a PUM under monitoring to another. For example, this can include identifying changes between a bevoken representing one state and another bevoken representing another state.

In some embodiments, such transition states may be represented by further bevokens, for example transition bevokens.

In some embodiments, there may be one or more routing algorithms, deployed for example by a care processing system, game management system, risk and/or safety management systems and/or other care village system, including for example those deployed on one or more sensors, device and/or system, where, for example, each decision that is an outcome of a game has, at least in part, one or more metrics, including for example, cost, such as resource, financial, temporal, power usage, privacy and/or other cost and/or potentially an optimized path to a further outcome, game, process and/or other operation. This can involve the identification of one or more patterns, which may be represented by a token, that represent the minimization or optimization of one or more of the metrics, including costs.

### Risk Assessment Games

One aspect of the system is the degree to which in any game the player(s) have complete or incomplete information. For example, if a player has complete information, then a Nash equilibrium can be achieved, though with incomplete information a Bayesian Nash equilibrium can be achieved, in both cases if the games are simultaneous. If the games are sequential the complete information yields a subgame of a perfect Nash equilibrium, whereas incomplete information yields a perfect Bayesian equilibrium.

This is, at least in part, based on rational responses for players as the game unfolds and as such involves consistency of a player's behavior. This approach is well suited to games involving sensors, devices and/or systems where their configuration is fundamentally based on a logical and rational set of specifications that determine their operations. In this example, the use of risk assessment games is well suited to this approach, such that outcome of such a game should be an equilibrium, and if not then this represents a risk that at least one of the players is not acting in a rational and/or consistent manner. For example, if a sensor, device and/or system is faulty and/or the stakeholders, including the PUM in an environment under monitoring are exhibiting behaviors that are neither consistent nor rational, then the risk game will produce an output representing such a risk. This can be in the form of a risk token which may then be evaluated by one or more other systems, including risk systems.

In some embodiments, such games may include one or more signals, represented by the strategy of the first player, for example a sensor, device and/or system and/or one or more stakeholders. In this situation, the subsequent players have, at least in part, some knowledge of the initial players strategy and consequently may invoke their own strategies to create an equilibrium. However, if any of the players strategies is inconsistent or lacks rationality, then the game will not resolve into equilibrium, but will resolve into a state that indicates a risk to the operations and/or behaviors of at least one of the players.

For example, an event may determine the strategy employed by one or more sensor, device and/or system, where such event is anomalous in the context of monitoring a PUM in an environment. For example, if an insect lands on a sensor, this can cause the sensor to generate a data token representing this event, which can determine the strategy employed by the sensors, device and/or system within the unfolding game. In this case the strategy employed by such a sensor, device and/or system may not represent consistent and rational behavior in the context of this and/or previous games.

In some embodiments, there may be risk module and/or system which operates a set of games to evaluate risks for a PUM in an environment. This risk system and/or module may use sets of historical data, such as the sequences of quiescent and event tokens that have been generated by one or more sensors, including situations where differing sensors have generated conflicting tokens and including, for example the use of models generated by one or more ML/AI module. For example, if a sensor generates a quiescent token and a second sensor generates an event token, where both sensors are monitoring the same PUM in an environment at the same time, then a module, such as for example a risk module can then evaluate the contradictory data. This evaluation can use one or more games.

In some embodiments the contradiction may involve the two or more tokens forming part of one or more games, where the payoffs for those games are further tokens, for example tokens that include configuration data for the one or more sensors undertaking the monitoring, including those that originally provided the contradictory data and/or other sensors that are located to monitor the PUM in an environment.

These configuration tokens may form part of a set of tokens that are used in one or more games that are played in parallel with the sensor-based games, in one or more digital twins. For example, a digital twin may have the same tokens as the physical sensors, and may on receiving the contradictory tokens apply one or more configuration tokens to the one or more sensors represented in that digital twin, through for example, playing multiple games, to determine, at least in part, the optimum configuration token, or set thereof, to be dispatched to the one or more sensors.

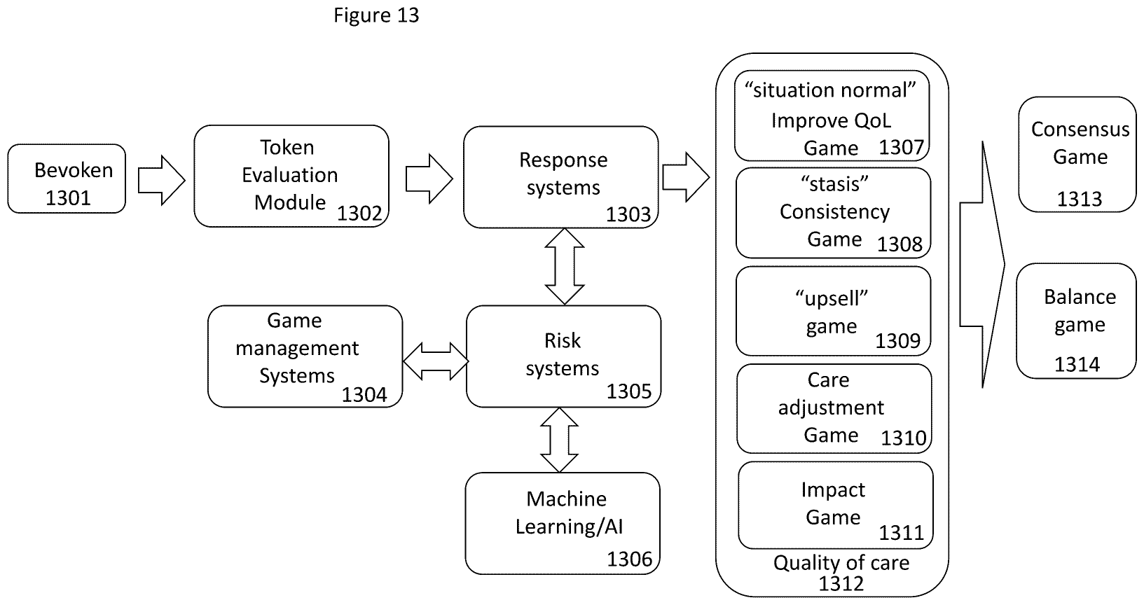

FIG. 13 illustrates an example bevoken (1301) representing the behaviors of one or more stakeholder, for example a PUM, where such bevoken is quiescent and such bevoken is evaluated, at least in part, by a token evaluation module (1302), which then communicates with one or more response systems (1303). Such response systems may interact with one or more risk systems (1305), which may communicate with game management systems (1304) and/or machine learning/AI systems (1306), which can include one or more digital twins and/or LLMs or other generative AI modules. These interactions can cause response systems to vary the output of those systems, for example, if the response systems are configured to instantiate one or more games that focus on the quality of care (1312) of a stakeholder, usually a PUM. In this example one or more of a set of games may be deployed, including for example a situation normal game with payoffs that are focused on improving the QoL (quality of life) of a stakeholder (1307), a stasis game, which has strategies and payoffs focused on the consistency of the care being provided (1308), an upsell game where the strategies and payoffs are focused on upselling products and/or services to one or more stakeholder (1309), a care adjustment game, where the outcome of the game is a variation in the care received by the one or more stakeholders (1310) and/or an impact game where the outcomes, payoffs and/or strategies focus on the actual and/or potential impacts of the responses for the one or more stakeholders (1311). Any and/or all of these games (1312), may then be evaluated by one or more other games, such as consensus games (1313) and/or balance games (1314), to at least in part evaluate the potential responses.

In some embodiments, such configuration may be determined, at least in part by one or more ML/AI modules, where such modules operate on a corpus of sensor, device and/or system and/or configuration of same data. Such an ML/AI module may also be used to, at least in part, determine the optimum configurations for one or more sensors using one or more games, where such ML/AI module acts as player on behalf of one or more sensors, devices and/or systems and/or operates as a separate player, such as one using thresholds or other data tokens.

In some embodiments where, for example the outcome of a game representing an event and/or change in state of a PUM is identified as having a significant detrimental wellness and health impact, for example a heart attack or the like, there may be one or more risk variables employed within the game representing these potential states. This can include risk management evaluations of the potential impact of the response to such events.

### Event Games

In some embodiments there may be games that are deployed one, before or during an event. These games may involve one or more time bases, representing, the time of an event start, stop and/or duration. In may circumstances such games may be invoked directly or indirectly by one or more sensors, devices and/or systems that generate one or more data sets, which may be reprehended by tokens, that represent the state of a PUM and/or other stakeholders in an environment.

One aspect of this approach is the identification of behavior characteristics, including patterns, that are representative of the “leading edge” of a particular behaviors, for example one represented by a bevoken. This can include the identification of the one or more patterns that form a bevoken, which may involve at least in part one or more games. These patterns may be evaluated by one or more matching module and may involve the use of games as matching mechanisms based on data set characteristics.

These event games may incorporate, directly or indirectly one or more risk metrics and/or variables, for example those that represent risk negotiation and/or resolution variables representing differing stakeholder incentives.

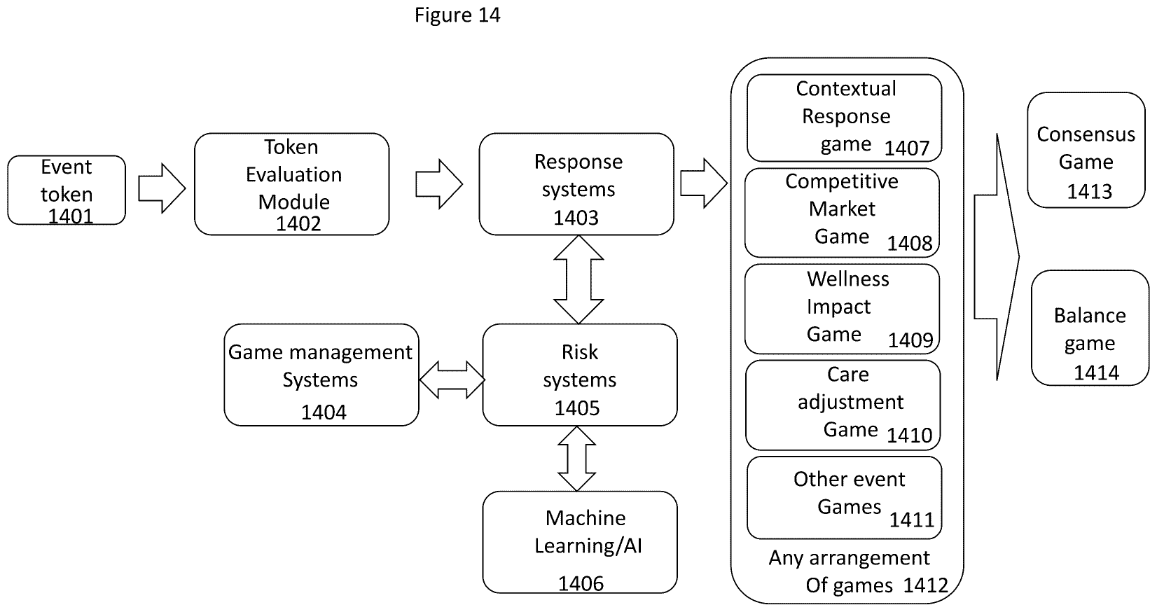

FIG. 14 Illustrates an event token (1401) which is communicated to a token evaluation module (1402), which then communicated the data set, at least in part, represented by the token (1401) to one or more response systems (1403). Such response systems (1403) can generate data sets that are communicated to one more arrangements of games (1412). For example, games that are responsive to events represented by tokens (1401), may include contextual response games (1407), competitive market games (1408), wellness impact games (1409), care adjustment games (1410) and/or other event games (1411). These games may interact with other games, for example, consensus game (1413) and/or balance games (1414) in any arrangement. The response systems may interact with risk systems (1405), which may also interact with game management systems (1404) and/or machine learning systems (1406), which may include one or more AI systems, for example LLM's and/or other generative AIU systems and can include one or more digital twins.

### Audit Games

In some embodiments there may be an audit module responsible for creating an audit trail for the operations of one or more sensors involved in the monitoring of a PUM, their environment and/or one or more other stakeholders interacting with such PUM and/or environment. One aspect of this audit module is the protection of the privacy of the PUM and/or other stakeholders whilst maintaining an accurate record of these operations and activities.

The data sets generated by the one or more sensors, devices and/or systems may only be relevant to an audit trail in that they represent a deviance from the behaviors being monitored, other than representing those behaviors, for example as bevokens.

Inn some embodiments, one or more distributed ledgers may be used as a repository for audit data, for example as a token, which in turn represents a sequence of bevokens, ToD tokens and the like.

An audit game may, for example, involve a payoff where the writing of a token to a repository, for example a distributed ledger, is validated as a complete and accurate record of a set of behaviors of a PUM and/or other stakeholders in any arrangement.

### Other Games

Care village systems may include games that are specialized to identify one or more behaviors of one or more stakeholders and/or operations of one or more sensors, devices and/or systems within the care village. Examples of these games are described herein, however there can be further games deployed to meet circumstances within the care village. In some embodiments this can include games identified, at least in part, by one or more machine learning modules.

Tacit collusion games can be used to identify and/or determine the quality and/or pricing of wellness services and/or products, where such quality and/or pricing of the such products and/or services is the result of two or more stakeholders colluding to set such quality and/or pricing variables. Such games may be invoked by suppliers of such services and/or products and/or by users of such, including the PUM. In this example the strategies deployed by the respective stakeholders may vary the outcomes of such games. In one example where each of the respective stakeholders, for example a supplier and a user, are using the same game, with differing strategies, these may form part of a multistage game to establish a mutually agreed outcome. In a further example, these games and their strategies may be evaluated by one or more machine learning and/or AI module so as to determine outcomes that represent the intentions of the stakeholders involved and may, for example, be used to determine any misalignment of such incentives, such as collusion between stakeholders and the like.

Matching games may support matching of one or more services and/or products to one or more stakeholders. For example, the stated or inherent needs of a stakeholder may be matched to an appropriate supplier of a product and/or service. This matching may, at least on part be enabled by the characteristics of the entities to be matched, which may be in form of specifications, for example metadata, attributes and the like. This can include the matching of a stakeholder to another. Such as for example, a carer to a PUM.

One version of the matching games involves pattern matching games, where for example, a repository of known and stored patterns can be compared to patterns detected by one or more sensors, devices and/or systems. In some embodiments, one or machine learning and/or AI system may be deployed to evaluate, coupled with one or more machine learning and/or AI module, to define optimum strategies for such matching.

In some embodiments, the use of one or more data evaluation games, for example those where the players have differing incentives, such as accuracy, speed, efficiency, cost and/or the like, may be deployed by one or more sensors, devices and/or systems. This can include games that identify one or more patterns that incorporate this data. These and other game types may include assistance by one or more machine learning and/or AI systems in any arrangement. For example, a machine learning and/or AI system, potentially in combination with one or more digital twin, may be configured to optimize one or more characteristic of these games, their outcomes and/or the strategies employed.

For example, this can include one or more behavior games with the purpose of identifying one or more predicates from patterns and the date sets comprising such patterns. For example, if a sensor, device and/or system generates a particular data set that has a reliable and repeatable relationship with an action, event or other behavior characteristic of one or more stakeholder, such a data set may be identified as a predicate to that pattern and/or behavior. Such an identification may have one or more attributes, such as a value as to the certainty of that relationship, determined at least in part by one or more machine learning module.

Such an approach may be deployed in games that are employed to determine any faults with the sensors, devices and/or systems, such that a data set is evaluated to identify any variance from the configuration of the sensors, device and/or system. For example, the sensors, device and/or system may be configured in manner that produces a specific data set under specific conditions, such as time, environment (heat, humidity, light and the like), and as such if the data set is inconsistent with the configuration, represented for example as game deployed on the sensor, device and/or system, then the outcome of the game is a fault event.

In some embodiments, there may be one or more balance games, which are typically multi part and/or multistage games, that at least in part, identify the relationships between the outcomes of other games. This can include interactions with systems and/or modules such as risk assessment systems, care processing systems, response systems and/or one or more machine learning and/or AI modules.

A further aspect is the use of games and game theory, embodied in for example, a game management system, which interacts with one or more machine learning and/or AI modules, where using one or more digital twins, responses, determined at least in part, through the one or more games deployed for the purpose, can be evaluated for their impact on the one or more stakeholders such responses are directed at. For example, if a response is to increase or decrease the time spent with a PUM by a carer, this may be evaluated in light of the potential impact on that PUM's behavior, as represented, for example, by one or more tokens. For example, if there is a correlation between the time spent by a carer and the time a PUM spends on their personal hygiene, the increase or reduction in the carer time on site may incorporate such a correlation, and for example extend the time spent by the carer which can involve a consequential increase in the cost of the carer to the PUM.

The deployment of multivariate response analytics can include the use of one or more machine learning systems to determine, at least in part, the correlations between the behaviors of the PUM and/or other stakeholders, so as to create, for example matrix model of these interdependencies, such that responses may be tailored to the individual requirements of each PUM so as to enhance their care and wellness.

In some embodiments, machine learning systems, such as those employing generative AI, may be configured to identify predicates, actions, events, messages and/or other communications that can influence one or more stakeholders. This can include the provision of incentives to one or more stakeholders, including in the form of messages or other communications that may influence their choices in their interactions with other stakeholders. These messages and/or communications may include actions, such as rewards, events and/or data, with the purpose of persuading the receiving stakeholder to undertake a behavior that has a positive impact on the PUM.

The previous description of the embodiments is provided to enable any person skilled in the art to practice the disclosure. Thus, the present disclosure is not intended to be limited to the embodiments shown herein but is to be accorded the widest scope consistent with the principles and features disclosed herein.

What is claimed is:

1. A system to monitor a person under care by one or more stakeholders, comprising:
a transceiver configured to receive a plurality of tokens from a plurality of environmental sensors configured to monitor the person under care, each of the tokens comprising a detected data set representing behaviors of the person under care in an environment, each of the behaviors is represented by a multi-dimensional feature set forming part of a health care profile for the person under care;
a non-transitory computer-readable storage medium configured to store a digital twin of the plurality of tokens from the plurality of environmental sensors, the digital twin comprising a dynamic tokenized representation of the multi-dimensional feature set forming part of a health care profile of the person under care in the environment;
at least one processor configured to evaluate the digital twin of the plurality of tokens from the plurality of environmental sensors as part of at least one game to optimize a set of configurations of the plurality of environmental sensors; and
change at least one state of the plurality of environmental sensors based on the game or notify the stakeholder.

2. The system of claim 1, wherein the at least one processor is further configured to identify potential future states of the person under care by analyzing the digital twin.

3. The system of claim 2, wherein the at least one processor is further configured to identify potential future states of the person under care by analyzing the digital twin and the plurality of tokens.

4. The system of claim 3, wherein the processor uses machine learning to analyze the digital twin and the plurality of tokens.

5. The system of claim 4, wherein the encryption key is unique to the person under care.

6. The system of claim 5, wherein the changing the state of the plurality of environmental sensors alters a monitoring focus of the environmental sensors.

7. The system of claim 6, wherein the monitoring focus increases the fidelity or granularity of the environmental sensors.

8. The system of claim 7, wherein the detected data set is from a breathing sensor, or heart-rate sensor.

9. The system of claim 1, wherein the transceiver is configured to transmit the token to a second care system.

10. The system of claim 9, wherein the second care system is determined based in part on the detected data set.

11. The system of claim 10, wherein the second care system is determined based in part on the at least one stakeholder's relationship to the person under care.

12. The system of claim 11, wherein the at least one stakeholder is determined by data pertinent to the at least one stakeholder's relationship with the person under care.

13. A system to monitor a person under care by one or more stakeholders, comprising:
a transceiver configured to receive a plurality of tokens from a plurality of environmental sensors configured to monitor the person under care, each of the tokens comprising a detected data set representing behaviors of the person under care in an environment, each of the behaviors is represented by a multi-dimensional feature set forming part of a health care profile for the person under care;
a non-transitory computer-readable storage medium configured to store a strategy in a game of optimizing the plurality of environmental sensors configured to monitor the person under care, the strategy having at least one strategy equilibrium;
at least one machine learning processor configured to apply the strategy to the plurality of tokens from the plurality of environmental sensors as part of the game to optimize set of configurations of the plurality of environmental sensors; and
change a state of the plurality of environmental sensors based on the game or notify the stakeholder.

14. The system of claim 13, wherein the at least one processor is further configured to identify potential future states of the person under care by analyzing the digital twin.

15. The system of claim 14, wherein the at least one processor is further configured to identify potential future states of the person under care by analyzing the digital twin and the plurality of tokens.

16. The system of claim 15, wherein the processor uses machine learning to analyze the digital twin and the plurality of tokens.

17. The system of claim 16, wherein the encryption key is unique to the person under care.

18. The system of claim 17, wherein the changing the state of the plurality of environmental sensors alters a monitoring focus of the environmental sensors.

19. The system of claim 18, wherein the monitoring focus increases the fidelity or granularity of the environmental sensors.
